SEQUENCE LISTING (135 sequences)

SEQ ID NO: 1 - DNA, 30 bp, Artificial Sequence
  Description of Artificial Sequence Synthetic anti-connexin oligonucleotide
gtaattgcggcaagaagaattgtttctgtc

SEQ ID NO: 2 - DNA, 30 bp, Artificial Sequence
  Description of Artificial Sequence Synthetic anti-connexin oligonucleotide
gtaattgcggcaggaggaattgtttctgtc

SEQ ID NO: 3 - DNA, 30 bp, Artificial Sequence
  Description of Artificial Sequence Synthetic anti-connexin oligonucleotide
ggcaagagacaccaaagacactaccagcat

SEQ ID NO: 4 - DNA, 27 bp, Artificial Sequence
  Description of Artificial Sequence Synthetic anti-connexin oligonucleotide
tcctgagcaatacctaacgaacaaata

SEQ ID NO: 5 - DNA, 20 bp, Artificial Sequence
  Description of Artificial Sequence Synthetic anti-connexin oligonucleotide
catctccttggtgctcaacc

SEQ ID NO: 6 - DNA, 20 bp, Artificial Sequence
  Description of Artificial Sequence Synthetic anti-connexin oligonucleotide
ctgaagtcgacttggcttgg

SEQ ID NO: 7 - DNA, 21 bp, Artificial Sequence
  Description of Artificial Sequence Synthetic anti-connexin oligonucleotide
ctcagatagtggccagaatgc

SEQ ID NO: 8 - DNA, 20 bp, Artificial Sequence
  Description of Artificial Sequence Synthetic anti-connexin oligonucleotide
ttgtccaggtgactccaagg

SEQ ID NO: 9 - DNA, 25 bp, Artificial Sequence
  Description of Artificial Sequence Synthetic anti-connexin oligonucleotide
cgtccgagcccagaaagatgaggtc

SEQ ID NO: 10 - DNA, 19 bp, Artificial Sequence
  Description of Artificial Sequence Synthetic anti-connexin oligonucleotide
agaggcgcacgtgagacac

SEQ ID NO: 11 - DNA, 19 bp, Artificial Sequence
  Description of Artificial Sequence Synthetic anti-connexin oligonucleotide
tgaagacaatgaagatgtt

SEQ ID NO: 12 - DNA, 25 bp, Artificial Sequence
  Description of Artificial Sequence Synthetic anti-connexin oligonucleotide
tttcttttctatgtgctgttggtga

SEQ ID NO: 13 - protein, 396 aa, Homo sapiens
MSWSFLTRLLEEIHNHSTFVGKIWLTVLIVFRIVLTAVGGESIYYDEQSKFVCNTEQPGCENVCYDAFAPLSHVRFWVFQIILVATPSVMYLGYAIHKIAKMEHGEADKKAARSKPYAMRWKQHRALEETEEDNEEDPMMYPEMELESDKENKEQSQPKPKHDGRRRIREDGLMKIYVLQLLARTVFEVGFLIGQYFLYGFQVHPFYVCSRLPCPHKIDCFISRPTEKTIFLLIMYGVTGLCLLLNIWEMLHLGFGTIRDSLNSKRRELEDPGAYNYPFTWNTPSAPPGYNIAVKPDQIQYTELSNAKIAYKQNKANTAQEQQYGSHEENLPADLEALQREIRMAQERLDLAVQAYSHQNNPHGPREKKAKVGSKAGSNKSTASSKSGDGKNSVWI

SEQ ID NO: 14 - protein, 382 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 15 - protein, 11 aa, Unknown
  Description Synthetic connexin peptide
FEVAFLLIQWI

SEQ ID NO: 16 - protein, 11 aa, Unknown
  Description Synthetic connexin peptide
LLIQWYIGFSL

SEQ ID NO: 17 - protein, 16 aa, Unknown
  Description Synthetic connexin peptide
SLSAVYTCKRDPCPHQ

SEQ ID NO: 18 - protein, 12 aa, Unknown
  Description Synthetic connexin peptide
VDCFLSRPTEKT

SEQ ID NO: 19 - protein, 11 aa, Unknown
  Description Synthetic connexin peptide
SRPTEKTIFII

SEQ ID NO: 20 - protein, 13 aa, Unknown
  Description Synthetic connexin peptide
LGTAVESAWGDEQ

SEQ ID NO: 21 - protein, 12 aa, Unknown
  Description Synthetic connexin peptide
QSAFRCNTQQPG

SEQ ID NO: 22 - protein, 12 aa, Unknown
  Description Synthetic connexin peptide
QQPGCENVCYDK

SEQ ID NO: 23 - protein, 13 aa, Unknown
  Description Synthetic connexin peptide
VCYDKSFPISHVR

SEQ ID NO: 24 - protein, 38 aa, Unknown
  Description Synthetic connexin peptide
AAESVWGDEIKSSFICNTLQPGCNSVCYDHFFPISHVR

SEQ ID NO: 25 - protein, 12 aa, Unknown
  Description Synthetic connexin peptide
ESVWGDEKSSFI

SEQ ID NO: 26 - protein, 12 aa, Unknown
  Description Synthetic connexin peptide
ICNTLQPGCNSV

SEQ ID NO: 27 - protein, 12 aa, Unknown
  Description Synthetic connexin peptide
SVCYDHFFPISH

SEQ ID NO: 28 - protein, 25 aa, Unknown
  Description Synthetic connexin peptide
RLVKCEAFPCPNTVDCFVSRPTEKT

SEQ ID NO: 29 - protein, 12 aa, Unknown
  Description Synthetic connexin peptide
VKCEAFPCPNTV

SEQ ID NO: 30 - protein, 12 aa, Unknown
  Description Synthetic connexin peptide
VDCFVSRPTEKT

SEQ ID NO: 31 - protein, 13 aa, Unknown
  Description Synthetic connexin peptide
VCYDHFFPISHVR

SEQ ID NO: 32 - protein, 18 aa, Unknown
  Description Synthetic connexin peptide
VWGDEKSSFICNTLQPGY

SEQ ID NO: 33 - protein, 15 aa, Unknown
  Description Synthetic connexin peptide
DEKSSFICNTLQPGY

SEQ ID NO: 34 - protein, 11 aa, Unknown
  Description Synthetic connexin peptide
SRPTEKTVFTV

SEQ ID NO: 35 - protein, 7 aa, Unknown
  Description Synthetic connexin peptide
SRPTEKT

SEQ ID NO: 36 - protein, 12 aa, Unknown
  Description Synthetic connexin peptide
ICNTLQPGCNSV

SEQ ID NO: 37 - protein, 16 aa, Unknown
  Description Synthetic connexin peptide
FLDTLHVCRRSPCPHP

SEQ ID NO: 38 - protein, 18 aa, Unknown
  Description Synthetic connexin peptide
KRDPCHQVDCFLSRPTEK

SEQ ID NO: 39 - protein, 35 aa, Unknown
  Description Synthetic connexin peptide
KEVWGDEQADFVCNTLQPGCKNVCYDHYFPISHIR

SEQ ID NO: 40 - protein, 35 aa, Unknown
  Description Synthetic connexin peptide
QEVWGDEQEDFVCNTLQPGCKNVCYDHFFPVSHIR

SEQ ID NO: 41 - protein, 35 aa, Unknown
  Description Synthetic connexin peptide
EEVWDDEQKDFVCNTKQPGCPNVCYDEFFPVSHVR

SEQ ID NO: 42 - protein, 35 aa, Unknown
  Description Synthetic connexin peptide
ERVWGDEQKDFDCNTKQPGCTNVCYDNYFPISNIR

SEQ ID NO: 43 - protein, 35 aa, Unknown
  Description Synthetic connexin peptide
ERVWSDDHKDFDCNTRQPGCSNVCFDEFFPVSHVR

SEQ ID NO: 44 - protein, 35 aa, Unknown
  Description Synthetic connexin peptide
ESVWGDEKSSFICNTLQPGCNSVCYDQFFPISHVR

SEQ ID NO: 45 - protein, 35 aa, Unknown
  Description Synthetic connexin peptide
ESVWGDEQSDFECNTAQPGCTNVCYDQAFPISHIR

SEQ ID NO: 46 - protein, 35 aa, Unknown; identical to SEQ ID NO: 45
  Description Synthetic connexin peptide

SEQ ID NO: 47 - protein, 35 aa, Unknown
  Description Synthetic connexin peptide
RPVYQDEQERFVCNTLQPGCANVCYDVFSPVSHLR

SEQ ID NO: 48 - protein, 35 aa, Unknown
  Description Synthetic connexin peptide
ESAWGDEQSAFRCNTQQPGCENVCYDKSFPISHVR

SEQ ID NO: 49 - protein, 35 aa, Unknown
  Description Synthetic connexin peptide
EDVWGDEQSDFTCNTQQPGCBNVCYBRAFPISHIR

SEQ ID NO: 50 - protein, 35 aa, Unknown
  Description Synthetic connexin peptide
EAIYSDEQAKFTCNTRQPGCDNVCYDAFAPLSHVR

SEQ ID NO: 51 - protein, 35 aa, Unknown
  Description Synthetic connexin peptide
ESSWGDEQADFRCDTIQPGCQNVCTDQAFPISHIR

SEQ ID NO: 52 - protein, 36 aa, Unknown
  Description Synthetic connexin peptide
GESIYYDEQSKFVCNTEQPGCENVCYDAFAPLSHVR

SEQ ID NO: 53 - protein, 39 aa, Unknown
  Description Synthetic connexin peptide
MYVFYVMYDGFSMQRLVKCNAWPCPNTVDCFVSRPTEKT

SEQ ID NO: 54 - protein, 39 aa, Unknown
  Description Synthetic connexin peptide
MYVFYFLYNGYHLPWVLKCGIDPCPNLVDCFISRPTEKT

SEQ ID NO: 55 - protein, 39 aa, Unknown
  Description Synthetic connexin peptide
LYIFHRLYKDYDMPRVVACSVEPCPHTVDCYISRPTEKK

SEQ ID NO: 56 - protein, 40 aa, Unknown
  Description Synthetic connexin peptide
LYLLHTLWHGFNMPRLVQCANVAPCPNIVDCYIARPTEKK

SEQ ID NO: 57 - protein, 39 aa, Unknown
  Description Synthetic connexin peptide
LYVFHSFYPKYILPPVVKCHADPCPNIVDCFISKPSEKN

SEQ ID NO: 58 - protein, 39 aa, Unknown
  Description Synthetic connexin peptide
MYVFYLLYPGYAMVRLVKCDVYPCPNTVDCFVSRPTEKT

SEQ ID NO: 59 - protein, 32 aa, Unknown
  Description Synthetic connexin peptide
LYGWTMEPVFVCQRAPCPYLVDCFVSRPTEKT

SEQ ID NO: 60 - protein, 32 aa, Unknown; identical to SEQ ID NO: 59
  Description Synthetic connexin peptide

SEQ ID NO: 61 - protein, 38 aa, Unknown
  Description Synthetic connexin peptide
GALHYFLFGFLAPKKFPCTRPPCTGVVDCYVSRPTSKS

SEQ ID NO: 62 - protein, 38 aa, Unknown
  Description Synthetic connexin peptide
LLIQWYIYGFSLSAVYTCKRDPCPHQVDCFLSRPTEKT

SEQ ID NO: 63 - protein, 38 aa, Unknown
  Description Synthetic connexin peptide
IAGQYFLYGFELKPLYRCDRWPCPNTVDCFISRPTEKT

SEQ ID NO: 64 - protein, 38 aa, Unknown
  Description Synthetic connexin peptide
LVGQYLLYGFEVRPFFPCSRQPCPHVVDCFVSRPTEKT

SEQ ID NO: 65 - protein, 38 aa, Unknown
  Description Synthetic connexin peptide
IVGQYFIYGIFLTTLHVCRRSPCPHPVNCYVSRPTEKN

SEQ ID NO: 66 - protein, 38 aa, Unknown
  Description Synthetic connexin peptide
LIGQYFLYGFQVHPFYVCSRLPCHPKIDCFISRPTEKT

SEQ ID NO: 67 - protein, 42 aa, Unknown
  Description Synthetic connexin peptide
LLIQWYIYGFSLSAVYTCKRDPCPHQVDCFLSRPTEKTIFII

SEQ ID NO: 68 - protein, 43 aa, Unknown
  Description Synthetic connexin peptide
MYVFYVMYDGFSMQRLVKCNAWPCPNTVDCFVSRPTEKTVFTV

SEQ ID NO: 69 - protein, 42 aa, Unknown
  Description Synthetic connexin peptide
YVFYFLYNGYHLPWVLKCGIDPCPNLVDCFISRPTEKTVFTI

SEQ ID NO: 70 - protein, 43 aa, Unknown
  Description Synthetic connexin peptide
LYIFHRLYKDYDMPRVVACSVEPCPHTVDCYISRPTEKKVFTY

SEQ ID NO: 71 - protein, 42 aa, Unknown
  Description Synthetic connexin peptide
LYLLHTLWHGFNMPRLVQCANVAPCPNIVDCYIARPTEKKTY

SEQ ID NO: 72 - protein, 43 aa, Unknown
  Description Synthetic connexin peptide
LYVFHSFYPKYILPPVVKCHADPCPNIVDCFISKPSEKNIFTL

SEQ ID NO: 73 - protein, 43 aa, Unknown
  Description Synthetic connexin peptide
MYVFYLLYPGYAMVRLVKCDVYPCPNTVDCFVSRPTEKTVFTV

SEQ ID NO: 74 - protein, 36 aa, Unknown
  Description Synthetic connexin peptide
LYGWTMEPVFVCQRAPCPYLVDCFVSRPTEKTIFII

SEQ ID NO: 75 - protein, 36 aa, Unknown; identical to SEQ ID NO: 74
  Description Synthetic connexin peptide

SEQ ID NO: 76 - protein, 42 aa, Unknown
  Description Synthetic connexin peptide
GALHYFLFGFLAPKKFPCTRPPCTGVVDCYVSRPTEKSLLML

SEQ ID NO: 77 - protein, 42 aa, Unknown
  Description Synthetic connexin peptide
IAGQYFLYGFELKPLYRCDRWPCPNTVDCFISRPTEKTIFII

SEQ ID NO: 78 - protein, 42 aa, Unknown
  Description Synthetic connexin peptide
LVGQYLLYGFEVRPFFPCSRQPCPHVVDCFVSRPTEKTVFLL

SEQ ID NO: 79 - protein, 41 aa, Unknown
  Description Synthetic connexin peptide
IVGQYFIYGIFLTTLHVCRRSPCPHPVNCYSRPTEKNVFIV

SEQ ID NO: 80 - protein, 42 aa, Unknown
  Description Synthetic connexin peptide
LIGQYFLYGFQVHPFYVCSRLPCHPKIDCFISRPTEKTIFLL

SEQ ID NO: 81 - protein, 45 aa, Unknown
  Description Synthetic connexin peptide
LGTAAESSWGDEQADFRCDTIQPGCQNVCTDQAFPISHIRFWVLQ

SEQ ID NO: 82 - protein, 14 aa, Unknown
  Description Synthetic connexin peptide
LGTAAESSWGDEQA

SEQ ID NO: 83 - protein, 13 aa, Unknown
  Description Synthetic connexin peptide
DEQADFRCDTIQP

SEQ ID NO: 84 - protein, 13 aa, Unknown
  Description Synthetic connexin peptide
TIQPGCQNVCTDQ

SEQ ID NO: 85 - protein, 13 aa, Unknown
  Description Synthetic connexin peptide
VCTDQAFPISHIR

SEQ ID NO: 86 - protein, 13 aa, Unknown
  Description Synthetic connexin peptide
AFPISHIRFWVLQ

SEQ ID NO: 87 - protein, 47 aa, Unknown
  Description Synthetic connexin peptide
MEVGFIVGQYFIYGIFLTTLHVCRRSPCPHPVNCYVSRPTEKNVFIV

SEQ ID NO: 88 - protein, 11 aa, Unknown
  Description Synthetic connexin peptide
MEVGFIVGQYF

SEQ ID NO: 89 - protein, 12 aa, Unknown
  Description Synthetic connexin peptide
IVGQYFIYGIFL

SEQ ID NO: 90 - protein, 14 aa, Unknown
  Description Synthetic connexin peptide
GIFLTTLHVCRRSP

SEQ ID NO: 91 - protein, 12 aa, Unknown
  Description Synthetic connexin peptide
RRSPCPHPVNCY

SEQ ID NO: 92 - protein, 12 aa, Unknown
  Description Synthetic connexin peptide
VNCYVSRPTEKN

SEQ ID NO: 93 - protein, 11 aa, Unknown
  Description Synthetic connexin peptide
SRPTEKNVFIV

SEQ ID NO: 94 - protein, 46 aa, Unknown
  Description Synthetic connexin peptide
LTAVGGESIYYDEQSKFVCNTEQPGCENVCYDAFAPLSHVRFWVFQ

SEQ ID NO: 95 - protein, 15 aa, Unknown
  Description Synthetic connexin peptide
LTAVGGESIYYDEQS

SEQ ID NO: 96 - protein, 13 aa, Unknown
  Description Synthetic connexin peptide
DEQSKFVCNTEQP

SEQ ID NO: 97 - protein, 13 aa, Unknown
  Description Synthetic connexin peptide
TEQPGCENVCYDA

SEQ ID NO: 98 - protein, 13 aa, Unknown
  Description Synthetic connexin peptide
VCYDAFAPLSHVR

SEQ ID NO: 99 - protein, 12 aa, Unknown
  Description Synthetic connexin peptide
APLSHVRFWVFQ

SEQ ID NO: 100 - protein, 47 aa, Unknown
  Description Synthetic connexin peptide
FEVGFLIGQYFLYGFQVHPFYVCSRLPCHPKIDCFISRPTEKTIFLL

SEQ ID NO: 101 - protein, 11 aa, Unknown
  Description Synthetic connexin peptide
FEVGFLIGQYF

SEQ ID NO: 102 - protein, 12 aa, Unknown
  Description Synthetic connexin peptide
LIGQYFLYGFQV

SEQ ID NO: 103 - protein, 14 aa, Unknown
  Description Synthetic connexin peptide
GFQVHPFYVCSRLP

SEQ ID NO: 104 - protein, 12 aa, Unknown
  Description Synthetic connexin peptide
SRLPCHPKIDCF

SEQ ID NO: 105 - protein, 12 aa, Unknown
  Description Synthetic connexin peptide
IDCFISRPTEKT

SEQ ID NO: 106 - protein, 11 aa, Unknown
  Description Synthetic connexin peptide
SRPTEKTIFLL

SEQ ID NO: 107 - protein, 11 aa, Unknown
  Description Synthetic connexin peptide
SRGGEKNVFIV

SEQ ID NO: 108 - protein, 10 aa, Unknown
  Description Synthetic connexin peptide
YVCSRLPCHP

SEQ ID NO: 109 - protein, 11 aa, Unknown
  Description Synthetic connexin peptide
QVHPFYVCSRL

SEQ ID NO: 110 - protein, 13 aa, Unknown
  Description Synthetic connexin peptide
FEVGFLIGQYFLY

SEQ ID NO: 111 - protein, 12 aa, Unknown
  Description Synthetic connexin peptide
GQYFLYGFQVHP

SEQ ID NO: 112 - protein, 12 aa, Unknown
  Description Synthetic connexin peptide
GFQVHPFYVCSR

SEQ ID NO: 113 - protein, 12 aa, Unknown
  Description Synthetic connexin peptide
AVGGESIYYDEQ

SEQ ID NO: 114 - protein, 12 aa, Unknown
  Description Synthetic connexin peptide
YDEQSKFVCNTE

SEQ ID NO: 115 - protein, 12 aa, Unknown
  Description Synthetic connexin peptide
NTEQPGCENVCY

SEQ ID NO: 116 - protein, 12 aa, Unknown
  Description Synthetic connexin peptide
CYDAFAPLSHVR

SEQ ID NO: 117 - protein, 12 aa, Unknown
  Description Synthetic connexin peptide
FAPLSHVRFWVF

SEQ ID NO: 118 - protein, 5 aa, Unknown
  Description Synthetic connexin peptide
LIGQY

SEQ ID NO: 119 - protein, 5 aa, Unknown
  Description Synthetic connexin peptide
QVHPF

SEQ ID NO: 120 - protein, 5 aa, Unknown
  Description Synthetic connexin peptide
YVCSR

SEQ ID NO: 121 - protein, 5 aa, Unknown
  Description Synthetic connexin peptide
SRLPC

SEQ ID NO: 122 - protein, 5 aa, Unknown
  Description Synthetic connexin peptide
LPCHP

SEQ ID NO: 123 - protein, 5 aa, Unknown
  Description Synthetic connexin peptide
GESIY

SEQ ID NO: 124 - protein, 6 aa, Unknown
  Description Synthetic connexin peptide
YDEQSK

SEQ ID NO: 125 - protein, 6 aa, Unknown
  Description Synthetic connexin peptide
SKFVCN

SEQ ID NO: 126 - protein, 8 aa, Unknown
  Description Synthetic connexin peptide
TEQPGCEN

SEQ ID NO: 127 - protein, 8 aa, Unknown
  Description Synthetic connexin peptide
VCYDAFAP

SEQ ID NO: 128 - protein, 10 aa, Unknown
  Description Synthetic connexin peptide
LSHVRFWVFQ

SEQ ID NO: 129 - protein, 14 aa, Unknown
  Description Synthetic connexin peptide
LIQYFLYGFQVHPF

SEQ ID NO: 130 - protein, 13 aa, Unknown
  Description Synthetic connexin peptide
VHPFYCSRLPCHP

SEQ ID NO: 131 - protein, 22 aa, Unknown
  Description Synthetic connexin peptide
VGGESIYYDEQSKFVCNTEQPG

SEQ ID NO: 132 - protein, 22 aa, Unknown
  Description Synthetic connexin peptide
TEQPGCENVCYDAFAPLSHVRF

SEQ ID NO: 133 - protein, 14 aa, Unknown
  Description Synthetic connexin peptide
AFAPLSHVRFWVFQ

SEQ ID NO: 134 - DNA, 1314 bp, Homo sapiens
ggcttttagcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtggtgcccaggcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctag

SEQ ID NO: 135 - DNA, 1149 bp, Homo sapiens
atgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctag

(19) United States
(12) Patent Application Publication
(10) Pub. No.: US 2011/0243964 A1
(43) Pub. Date: Oct. 6, 2011

(54) TREATMENT OF ORTHOPEDIC CONDITIONS

(75) Inventor: Bradford James Duft, Rancho Santa Fe, CA (US)

(73) Assignee: BRADFORD JAMES DUFT, RANCHO SANTA FE, CA (US)

(21) Appl. No.: 12/809,980

(22) PCT Filed: Dec. 22, 2008
(86) PCT No.: PCT/US08/14022

§ 371 Date: Jun. 21, 2010

Related U.S. Application Data
(60) Provisional application No. 61/008,834, filed on Dec. 21, 2007.

(51) Int. Cl. A61K 38/17; A61K 31/7088; A61K 38/10; A61K 38/08; A61K 39/395; A61P 19/02; A61P 29/00

(52) U.S. Cl. 424/172.1; 514/44 A; 514/21.5; 514/21.6; 514/21.4; 514/21.2

(57) ABSTRACT

Methods, compounds, compositions, kits and articles of manufacture comprising anti-connexin agents for treatment of orthopedic disorders, diseases, or conditions, and improving recoveries and outcomes of orthopedic procedures or surgeries.

This application is a National Stage Application under 35 U.S.C. §371 of International Application No. PCT/US2008/014022, filed on Dec. 22, 2008 which claims the benefit of priority to U.S. Provisional Application No. 61/008,834 filed on Dec. 21, 2007. The disclosures of both are incorporated herein by reference.

### FIELD

The inventions relate to orthopedic diseases, disorders and conditions and methods of treatment thereof and related pharmaceutical compositions, formulations, articles of manufacture and kits comprising such compositions.

### BACKGROUND

The following includes information that may be useful in understanding the present invention. It is not an admission that any of the information provided herein is prior art, or relevant, to the presently described or claimed inventions, or that any publication or document that is specifically or implicitly referenced is prior art.

In humans and other mammals wound injury triggers an organized complex cascade of cellular and biochemical events that will in most cases result in a healed wound. An ideally healed wound is one that restores normal anatomical structure, function, and appearance at the cellular, tissue, organ, and organism levels. Wound healing, whether initiated by surgery, trauma, microbes or foreign materials, proceeds via a complex process encompassing a number of overlapping phases, including inflammation, epithelialization, angiogenesis and matrix production and deposition. Normally, these processes lead to a mature wound and a certain degree of scar formation. Although inflammation and repair mostly occur along a prescribed course, the sensitivity of the process is dependent on the balance of a variety of wound healing modulating factors, including for example, a network of regulatory cytokines and growth factors.

Normal function of a joint and its movement can be severely impaired by inflammation, scarring and/or abnormal tissue formation that takes place both inside and/or around the joint (intra-articular or peri-articular) or any other affected area during the subsequent wound healing process following orthopedic surgical procedures. This may result in tenderness, aching, pain, and lengthy recovery times, as well as the loss of joint mobility or reduced range of motion, tonicity, or elasticity of the joint/articular structures, such as for example, muscle, tendon, capsule, bone, or ligament. Reduced joint mobility may also involve permanently altered or shortened joint or tissue architecture.

Altered or abnormal joint mobility or joint architecture may also be associated with or caused by a variety of injuries and conditions such as, for example, metabolic disorders, ischemia, trauma, injury to joint, capsule, bone, cartilage, tendon, ligament or muscle, fractures, subluxation, dislocation, crush injuries, prolonged immobilization (e.g., immobilization of a joint in a cast or splint), and paralysis.

To date, common surgical interventions to alleviate altered or abnormal joint mobility or joint architecture caused by scarring and abnormal tissue formation have been met with limited success as corrective surgical procedure is also a form of controlled injury or trauma and the procedure often ignites or reignites inflammation and proliferation in the tissue and the reformation of the scar and tissue abnormality around the point of articulation.

In certain cases, reduced joint motion may have hereditary components and the primary scar and/or the abnormal tissue growth may take place around and/or on the outside of the joint. One such condition is the Dupytren's contracture in which the connective tissue in the palmer aspect of the hand begins to scar and thicken and eventually leading to deformation of the hand at the site of the thickening, and the resultant loss of range of motion of the fingers. Treatment for contractures is limited to after a contracture is already established. Surgical release procedures include manipulation under anesthesia which involves putting the patient to sleep and breaking down the adhesion by forcing the joint (forced manipulation). This often reignites the inflammation and proliferation in the tissue and the reformation of the scar and stiffness. Surgical interventions may also include an open surgical procedure that involves releasing (open release) and removing the restricting scar and abnormal tissue (debulking). This operation may also be performed through an arthroscope, where the scar and restricting tissue is released and removed using special tools (athroscopic release). Unfortunately, surgical interventions fail, and may actually make the condition worse as the surgery itself is a controlled injury or trauma, which can cause even more scarring and abnormal tissue formation in response to the surgical injury. Treatments such as physiotherapy and range of motion exercises are used but with limited success. Pharmacological therapy has also been attempted with limited or no success. Agents most often used include non-steriodal anti-inflammatories, steroids and radiation.

It has been reported that abnormal connexin function may be linked to certain disease states (e.g. heart diseases) (A. C. de Carvalho, et al., J Cardiovasc Electrophysiol 1994, 5 686). In certain connexin proteins, alterations in the turnover and trafficking properties may be induced by the addition exogenous agents which may affect the level of gap junctional intercellular communication (Darrow, B. J., et al. (1995). Circ Res 76: 381; Lin R, et al. (2001) J Cell Biol 154(4):815). Gap junctions are cell membrane structures that facilitate direct cell-cell communication. A gap junction channel is formed of two connexins (hemichannels), each composed of six connexin subunits. Each hexameric connexon docks with a connexon in the opposing membrane to form a single gap junction. Gap junction channels are reported to be found throughout the body. Tissue such as the corneal epithelium, for example, has six to eight cell layers, yet expresses different gap junction channels in different layers with connexin 43 in the basal layer and connexin 26 from the basal to middle wing cell layers. In general, connexins are a family of proteins, commonly named according to their molecular weight or classified on a phylogenetic basis into alpha, beta, and gamma subclasses. At least 20 human and 19 murine isoforms have been identified. Different tissues and cell types are reported to have characteristic patterns of connexin protein expression and tissues such as cornea have been shown to alter connexin protein expression pattern following injury or transplantation (Qui, C. et al., (2003) Current Biology, 13:1967-1703; Brander et al., (2004), J. Invest Dermatol. 122:1310-20).

It has been reported that abnormal connexin function may be linked to certain disease states (e.g. heart diseases) (A. C. de Carvalho, et al., J Cardiovasc Electrophysiol 1994, 5 686). In certain connexin proteins, alterations in the turnover and trafficking properties may be induced by the addition exogenous agents which may affect the level of gap junctional intercellular communication (Darrow, B. J., et al. (1995). Circ Res 76: 381; Lin R, et al. (2001) J Cell Biol 154(4):815). Antisense technology has been reported for the modulation of the expression for genes implicated in viral, fungal and metabolic diseases. See, e.g., U.S. Pat. No. 5,166,195, (oligonucleotide inhibitors of HIV), U.S. Pat. No. 5,004,810 (oligomers for hybridizing to herpes simplex virus Vmw65 mRNA and inhibiting replication). See also U.S. Pat. No. 7,098,190 to Becker et al. (formulations comprising antisense nucleotides to connexins). Peptide inhibitors (including peptidomimetics) of gap junctions and hemichannels have been reported. See for example Berthoud, V. M. et al., Am J. Physiol. Lung Cell Mol. Physiol. 279: L619-L622 (2000); Evans, W. H. and Boitano, S. Biochem. Soc. Trans. 29: 606-612, and De Vriese A. S., et al. Kidney Int. 61: 177-185 (2001). See also Green and Becker, WO2006/134494 (“Anti-connexin compounds and methods of use”).

Despite advances in the understanding of the principles of the mechanisms underlying the wound healing processes associated with orthopedic procedures, there remains a significant unmet need for suitable therapeutic options for improving outcomes and recoveries.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

The invention generally relates to the use (including for use in treatment or in the manufacture or preparation of compositions, formulations, articles of manufacture, and kits) of one or more anti-connexin polynucleotides (for example, connexin inhibitors such as alpha-1 connexin oligodeoxynucleotides) in combination with one or more anti-connexin peptides, peptidomimetics (for example, alpha-1 anti-connexin peptides, peptidomimetics), and/or gap junction modifying agents (e.g. connexin carboxy-terminal polypeptides, including connexin carboxy-terminal polypeptides that block or inhibit or otherwise interfere with ZO-1 protein interaction or binding, and hemichannel closing compounds, including connexin phosphorylation compounds) for the treatment of a subject suffering from, predisposed to, or at risk for various orthopedic-related diseases, disorders, or conditions. Preferred anti-connexin polynucleotides (for example, antisense connexin polynucleotides), anti-connexin peptides, anti-connexin peptidomimetics, and gap junction modifying agents (e.g. connexin carboxy-terminal polypeptides, including connexin carboxy-terminal polypeptides that block or inhibit or otherwise interfere with ZO-1 protein interaction or binding, and hemichannel closing compounds, including connexin phosphorylation compounds), are anti-connexin 43 polynucleotides (for example, antisense connexin 43 polynucleotides), anti-connexin 43 peptides, anti-connexin 43 peptidomimetics, and connexin 43 gap junction modifying agents (e.g. connexin 43 carboxy-terminal polypeptides, including connexin 43 carboxy-terminal polypeptides that block or inhibit or otherwise interfere with ZO-1 protein interaction or binding, and connexin 43 hemichannel closing compounds, including connexin 43 phosphorylation compounds).

Compositions and methods of the invention for the treatment of a subject suffering from, predisposed to, or at risk for various orthopedic-related diseases, disorders, or conditions comprising administration of one or more anti-connexin peptides or peptidomimetics in combination with one or more gap junction modifying agents and/or one or more anti-connexin polynucleotides are disclosed and claimed.

Compositions and methods of the invention for the treatment of a subject during or following an orthopedic procedure or surgery comprising administration of one or more anti-connexin peptides or peptidomimetics in combination with one or more gap junction modifying agents and/or one or more anti-connexin polynucleotides are disclosed and claimed.

Compositions and methods of the invention for the treatment of a subject suffering from, predisposed to, or at risk for various orthopedic-related diseases, disorders, or conditions that employ a first anti-connexin agent in combination with a second anti-connexin agent are also disclosed and claimed. A first anti-connexin agent may be selected from the group consisting of anti-connexin oligonucleotides, anti-connexin peptides, anti-connexin peptidomimetics, gap junction closing compounds, hemichannel closing compounds, and connexin carboxy-terminal polypeptides. A second anti-connexin agent is selected from the above group as modified to subtract the subcategory of anti-connexin agents from which the first anti-connexin agent was selected.

Compositions and methods of the invention for the treatment of a subject during or following an orthopedic procedure or surgery that employ a first anti-connexin agent in combination with a second anti-connexin agent are also disclosed and claimed. A first anti-connexin agent may be selected from the group consisting of anti-connexin oligonucleotides, anti-connexin peptides, anti-connexin peptidomimetics, gap junction closing compounds, hemichannel closing compounds, and connexin carboxy-terminal polypeptides. A second anti-connexin agent is selected from the above group as modified to subtract the subcategory of anti-connexin agents from which the first anti-connexin agent was selected.

The invention includes a pharmaceutical composition comprising a pharmaceutically acceptable anti-connexin polynucleotide and a pharmaceutically acceptable anti-connexin peptide or peptidomimetic, for the treatment of a subject during or following an orthopedic procedure or surgery or suffering from, predisposed to, or at risk for various orthopedic-related diseases, disorders, or conditions. It also includes a pharmaceutical composition comprising a first anti-connexin agent and a second anti-connexin agent, wherein the first anti-connexin agent is selected from the group consisting of anti-connexin oligonucleotides, anti-connexin peptides, anti-connexin peptidomimetics, gap junction closing compounds, hemichannel closing compounds, and connexin carboxy-terminal polypeptides useful for treating a subject during or following an orthopedic procedure or surgery or suffering from, predisposed to or at risk of orthopedic diseases, disorders and/or condition, and the second anti-connexin agent is selected from the above group as modified to subtract the subcategory of anti-connexin agents from which the first anti-connexin agent was selected. Such formulations include, for example, topical, instillation, and injectable delivery forms and formulations. Such delivery forms and formulations include those for the treatment of a subject as disclosed herein. Preferred anti-connexin polynucleotides are anti-connexin 43 oligonucleotides (ODN). Preferred peptides, peptidomimetics, or gap junction modifying agents, are anti-connexin 43 peptides, peptidomimetics, or gap junction modifying agents, e.g., anti-connexin 43 hemichannel blocking peptides or anti-connexin 43 hemichannel blocking peptidomimetics. Preferred gap junction closing compounds and hemichannel closing compounds are connexin 43 gap junction closing compounds and connexin 43 hemichannel closing compounds. Preferred connexin carboxy-terminal polypeptides are connexin 43 carboxy-terminal polypeptides.

Treatment of a subject during or following an orthopedic procedure or surgery, or a subject suffering from, predisposed to, or at risk for various orthopedic-related diseases, disorders, or conditions, with one or more pharmaceutical compositions of the invention, e.g. a peptide or peptidomimetic; e.g., an anti-connexin oligonucleotide (e.g., an anti-connexin ODN) and a connexin hemichannel blocking agent; e.g., a peptide or peptidomimetic, or a first anti-connexin agent and a second anti-connexin agent, may comprise their simultaneous, separate, sequential or sustained administration.

In one embodiment, a composition comprising one or more anti-connexin polynucleotides is administered at or about the same time as one or more anti-connexin peptides, peptidomimetics, or gap junction modifying agents. In one embodiment, a composition comprising one or more anti-connexin polynucleotides is administered within at least about thirty minutes of one or more anti-connexin peptides, peptidomimetics, or gap junction modifying agents. In one embodiment, a composition comprising one or more anti-connexin polynucleotides is administered within at least about one hour of one or more anti-connexin peptides, peptidomimetics, or gap junction modifying agents. In one embodiment, a composition comprising one or more anti-connexin polynucleotides is administered within at least about 2-12 hours of one or more anti-connexin peptides, peptidomimetics, or gap junction modifying agents. In one embodiment, a composition comprising one or more anti-connexin polynucleotides is administered within at least about 24-48 hours of one or more anti-connexin peptides, peptidomimetics, or gap junction modifying agents. In another embodiment the anti-connexin polynucleotide and anti-connexin peptide or peptidomimetic are administered within about 1-8 hours of each other, within about one day of each other, or within about one week of each other. Other embodiments include administration of one or more anti-connexin polynucleotides and/or one or more anti-connexin peptides, peptidomimetics, or gap junction modifying agents, and one or more gap junction closing compounds, one or more hemichannel closing compounds, and/or one or more connexin carboxy-terminal polypeptides.

The invention also includes pharmaceutical compositions, useful for the treatment of a subject during or following an orthopedic procedure or surgery or suffering from, predisposed to, or at risk for various orthopedic-related diseases, disorders, or conditions, comprising (a) a therapeutically effective amount of an anti-connexin peptide or pepidomimetic and (b) a therapeutically effective amount of an antisense polynucleotide to the mRNA of a connexin protein. Most preferably, this connexin is connexin 43. The invention also includes pharmaceutical compositions, useful for the treatment of a subject during or following an orthopedic procedure or surgery or suffering from, predisposed to, or at risk for various orthopedic-related diseases, disorders, or conditions, comprising (a) a therapeutically effective amount of an anti-connexin peptide or peptidomimetic and/or (b) and a therapeutically effective amount of one or more of a gap junction closing compound, a hemichannel closing compound, and a connexin carboxy-terminal polypeptide. Most preferably, in the case of gap junction modifying agents, for example, gap junction closing compound and hemichannel closing compounds useful, the gap junction or hemichannel is a connexin 43 gap junction or hemichannel. Most preferably, in the case of connexin carboxy-terminal polypeptides, the connexin is connexin 43. In other embodiments, sub-therapeutically effective amounts of two or more anti-connexin agents are used for administration separately or jointly to provide a combined action that is therapeutically effective.

Pharmaceutical compositions useful for the treatment of a subject during or following an orthopedic procedure or surgery or suffering from, predisposed to, or at risk for various orthopedic-related diseases, disorders, or conditions are also provided in the form of a combined preparation, for example, as an admixture of therapeutically effective amounts of two or more anti-connexin agents, for example one or more anti-connexin polynucleotides and one or more anti-connexin peptides, peptidomimetics, or gap junction modifying agents. In other embodiments, sub-therapeutically effective amounts of two or more anti-connexin agents are used in combination to provide a desired therapeutically effect.

The term “a combined preparation” includes a “kit of parts” in the sense that the combination partners as defined above can be dosed independently or by use of different fixed combinations with distinguished amounts of the combination partners (a) and (b), i.e. simultaneously, separately or sequentially. The parts of the kit can then, for example, be administered simultaneously or chronologically staggered, that is at different time points and with equal or different time intervals for any part of the kit of parts.

In one embodiment a combined preparation is administered, wherein two or more separate compositions are administered to a subject, wherein the first composition comprises a therapeutically effective amount of an anti-connexin 43 polynucleotide and the second composition comprises a therapeutically effective amount of an anti-connexin 43 peptide or peptidomimetic. In another embodiment a third composition is administered comprising therapeutically effective amounts of one or more anti-connexin polynucleotides, peptides, peptidomimetics, or gap junction modifying agents. The third composition may also comprise therapeutically effective amounts of one or more gap junction closing compounds, hemichannel closing compounds, or connexin carboxy-terminal polypeptides. In other embodiments, sub-therapeutically effective amounts of one or more anti-connexin 43 polynucleotides, anti-connexin 43 peptides or peptidomimetics, gap junction modifying agents, gap junction closing compounds, hemichannel closing compounds, and/or connexin carboxy-terminal polypeptides are used in combination to provide a desired therapeutically effect.

In one aspect, the invention includes pharmaceutical compositions useful for the treatment of a subject during or following an orthopedic procedure or surgery or suffering from, predisposed to, or at risk for various orthopedic-related diseases, disorders, or conditions, including topical delivery forms and formulations, comprising a pharmaceutically acceptable carrier and therapeutically effective amounts of an anti-connexin peptide, peptidomimetic alone or in combination with an anti-connexin oligonucleotide and/or a gap junction modifying agent. In another aspect, the invention includes pharmaceutical compositions useful for treatment of a subject during or following an orthopedic procedure or surgery or suffering from, predisposed to, or at risk for various orthopedic-related diseases, disorders, or conditions, including instillation or injectable delivery forms and formulations, comprising a pharmaceutically acceptable carrier and therapeutically effective amounts of an anti-connexin peptide, peptidomimetic alone or in combination with an anti-connexin oligonucleotide and/or a gap junction modifying agent.

In one aspect, the invention includes pharmaceutical compositions useful for the treatment of a subject during or following an orthopedic procedure or surgery or suffering from, predisposed to, or at risk for various orthopedic-related diseases, disorders, or conditions, including topical, instillation, and injectable delivery forms and formulations, comprising a pharmaceutically acceptable carrier and therapeutically effective amounts of a first anti-connexin agent and a second anti-connexin agent as described herein, for example, an anti-connexin polynucleotide and one or more anti-connexin peptides, peptidomimetics, or gap junction modifying agents. Examples of anti-connexin polynucleotides include anti-connexin oligodeoxynucleotides (“ODN”), including antisense (including modified and unmodified backbone antisense), RNAi, and siRNA. Suitable anti-connexin peptides include connexin binding peptides. Suitable anti-connexin agents include for example, antisense ODNs and other anti-connexin oligonucleotides, peptides and peptidomimetics against connexins 43, 26, 37, 30, and 31.1 and 32. In certain embodiments, suitable compositions include multiple anti-connexin agents in Combination, including for example, anti-connexin 43, 26, 30, and 31.1 agents. Preferred anti-connexin agents, including anti-connexin oligonucleotides and anti-connexin peptides and peptidomimetics, are directed against connexin 43.

The present invention provides methods for the treatment of a subject during or following an orthopedic procedure or surgery or suffering from, predisposed to, or at risk for various orthopedic-related diseases, disorders, or conditions through the use of two or more anti-connexin agents administered simulataneously, separate, or sequentially. In a preferred embodiment, the combined use of a first anti-connexin agent and a second anti-connexin agent as described herein, for example, one or more anti-connexin polynucleotides and one or more anti-connexin peptides, peptidomimetics, or gap junction modifying agents has an additive, synergistic or super-additive effect in the treatment of a subject suffering from, predisposed to, or at risk for various orthopedic-related diseases, disorders, or conditions. In a preferred embodiment, the administration of a combined preparation will have fewer administration time points and/or increased time intervals between administrations as a result of such combined use. In another preferred embodiment, the combined use of a first anti-connexin agent and a second anti-connexin agent as described herein, for example, one or more anti-connexin polynucleotides and one or more anti-connexin peptides, peptidomimetics, or gap junction modifying agents, allows a reduced frequency of administration. In another preferred embodiment, the combined use of a first anti-connexin agent and a second anti-connexin agent as described herein, for example, one or more anti-connexin polynucleotides and one or more anti-connexin peptides, peptidomimetics, or gap junction modifying agents, allows the use of reduced doses of such agents compared to the dose or doses that may be effective when the agent is administered alone. In general, these anti-connexin agent combinations will have improved therapeutic results over administration of single anti-connexin agents.

In another aspect, the invention includes methods for administering a therapeutically effective amount of a first anti-connexin agent and a second anti-connexin agent as described herein, for example, one or more anti-connexin polynucleotides and one or more anti-connexin peptides, peptidomimetics, or gap junction modifying agents, formulated in a delayed release preparation, a slow release preparation, an extended release preparation, a controlled release preparation, and/or in a repeat action preparation to a subject during or following an orthopedic procedure or surgery or suffering from, predisposed to, or at risk for various orthopedic-related diseases, disorders, or conditions.

In certain other aspects, the invention also relates to methods of treating a subject during or following an orthopedic procedure or surgery comprising administration of (a) a therapeutically effective amount of one or more anti-connexin peptides or peptidomimetics, alone or in combination with one or more gap junction modifying agents and (b) a therapeutically effective amount of one or more anti-connexin polynucleotides. In one embodiment, surgical outcome is improved. In one embodiment, administration is effective to decrease or prevent, in whole or in part, joint contraction in a post-operative subject. In one embodiment, administration is effective to improve recovery time in a post-operative subject. In one embodiment, administration is effective to decrease pain in a post-operative subject. In one embodiment, administration is effective to improve overall recovery result in a post-operative subject. In one embodiment, improved recovery results comprises increased post-operative mobility. In other embodiments, sub-therapeutically effective amounts of one or more anti-connexin polynucleotides and anti-connexin peptides or peptidomimetics, are administered alone or in combination to provide a desired therapeutic effect

In one aspect the invention relates to methods and compositions for decreasing post-orthopedic-surgical vascular damage at the surgical site comprising administration of (a) one or more anti-connexin peptides or peptidomimetics, alone or in combination with one or more gap junction modifying agents and (b) one or more anti-connexin polynucleotides.

In one aspect the invention relates to methods and compositions for improving or maintaining vascular integrity during and/or during or following an orthopedic-surgical or other procedure comprising administration of (a) a therapeutically effective amount of one or more anti-connexin peptides or peptidomimetics, alone or in combination with one or more gap junction modifying agents and (b) a therapeutically effective amount of one or more anti-connexin polynucleotides. In other embodiments, sub-therapeutically effective amounts of one or more anti-connexin polynucleotides, anti-connexin peptides or peptidomimetics, and/or gap junction modifying agents are used in alone or in combination to provide a desired therapeutic effect.

In one embodiment, the subject is treated during following one of the following surgical procedures: e.g., a release procedure, an arthroscopic procedure, a joint surgery (e.g., hip, shoulder or knee surgery, including replacement procedures). In general, orthopedic surgeries addressed with the inventions described and claimed herein include hand surgery; shoulder and elbow surgery; total joint reconstruction (arthroplasty); foot and ankle surgery; spine surgery; surgical sports medicine; and orthopedic trauma. Thus, for example, orthopedic surgeries include knee arthroscopy and meniscectomy; shoulder arthroscopy and decompression; carpal tunnel release; knee arthroscopy and chondroplasty; removal of support implants; knee arthroscopy and anterior cruciate ligament reconstruction; knee replacement; repair of femoral neck fractures; repair of trochanteric fractures; debridement of skin/muscle/bone/fracture; knee arthroscopy repair of both menisci; hip replacement; shoulder arthroscopy/distal clavicle excision; repair of rotator cuff tendon; repair fracture of radius/ulna; laminectomy; repair of ankle fracture (bimalleolar type); shoulder arthroscopy and débridement; lumbar spinal fusion; repair fracture of the distal radius; low back intervertebral disc surgery; incise finger tendon sheath; repair of ankle fracture (fibula); repair of femoral shaft fracture; repair of trochanteric fracture. Total hip replacement, total shoulder replacement, and total knee replacement are included as well, as is uni-compartment knee replacement, in which only one side of an arthritic knee is replaced, and joint replacements for other joints, including elbow, wrist, ankle, and fingers. Also included in orthopedic surgeries is bone grafting, a surgical procedure that replaces missing bone with material from the patient's own body, or an artificial, synthetic, or natural substitute.

Arthroscopic applications, topical applications, injections and instillations are also included within the invention.

In yet another aspect, the invention provides a method of treating a subject during or following an orthopedic procedure or surgery or suffering from, predisposed to, or at risk for various orthopedic-related diseases, disorders, or conditions comprising administering to a subject in need thereof a first composition and a second composition, said first composition comprising a therapeutically effective amount of a anti-connexin 43 polynucleotide and said second composition comprising a therapeutically effective amount of an anti-connexin 43 peptide or peptidomimetic. In one embodiment the first composition is administered first. In another embodiment, the second composition is administered first. In a further embodiment, the method further comprises administration of a third composition, wherein the third composition comprises an anti-connexin polynucleotide, peptide, peptidomimetic or gap junction modifying agent. In one embodiment the third composition is administered first.

In one aspect, the invention provides a method for preventing and/or decreasing a joint contracture during or following an orthopedic procedure or surgery, comprising administering to a subject in need thereof a therapeutically effective amount of a pharmaceutical composition comprising a first anti-connexin agent and a second anti-connexin agent as described herein, for example, one or more anti-connexin polynucleotides and one or more anti-connexin peptides, peptidomimetics, or gap junction modifying agents. In one embodiment, said method comprises administration of two pharmaceutical compositions, the first composition comprising a therapeutically effective amount of one or more anti-connexin polynucleotides and the second pharmaceutical composition comprising a therapeutically effective amount of one or more anti-connexin peptides, peptidomimetics, or gap junction modifying agents. In one embodiment the first composition is administered first. In another embodiment, the second composition is administered first. In a further embodiment, the method, further comprises administration of a third composition, wherein the third composition comprises a therapeutically effective amount of an anti-connexin polynucleotide, peptide or peptidomimetic. In one embodiment the third composition is administered first. In one embodiment the third composition is administered first. In one embodiment, the composition is administered to the site of the injury before, at the time of and/or after a release procedure (e.g., forced manipulation, open release, arthroscopic release, or debulking of scar) to prevent the recurrence of abnormal tissue and/or further contracture. In other embodiments, sub-therapeutically effective amounts of anti-connexin agents are used for administration separately or jointly to provide a combined action that is therapeutically effective.

In a further aspect, the invention includes dressings and matrices capable of delivering a therapeutically effective amount of an anti-connexin peptide (e.g., a hemichannel blocker), or a first anti-connexin agent and a second anti-connexin agent as described herein, for example, one or more anti-connexin polynucleotides and one or more anti-connexin peptides, peptidomimetics, or gap junction modifying agents to a subject a subject during or following an orthopedic procedure or surgery or suffering from, predisposed to, or at risk for various orthopedic-related diseases, disorders, or conditions.

In another aspect, the invention includes an article of manufacture comprising a vessel containing a therapeutically effective amount of an anti-connexin peptide (e.g., a hemichannel blocker), or therapeutically effective amounts of a first anti-connexin agent and a second anti-connexin agent as described herein, for example, one or more pharmaceutically acceptable anti-connexin polynucleotides and one or more pharmaceutically acceptable anti-connexin peptides, peptidomimetics, or gap junction modifying agents and instructions for use, including use for the treatment of a subject as described herein. In other embodiments, sub-therapeutically effective amounts of first and second anti-connexin agents are used to provide a desired therapeutic effect.

The invention includes an article of manufacture comprising packaging material containing one or more dosage forms containing a therapeutically effective amount of an anti-connexin peptide (e.g., a hemichannel blocker), or therapeutically effective amounts of a first anti-connexin agent and a second anti-connexin agent as described herein, for example, one or more anti-connexin polynucleotides and one or more anti-connexin peptides, peptidomimetics, or gap junction modifying agents, wherein the packaging material has a label that indicates that the dosage form can be used for a subject during or following an orthopedic procedure or surgery or suffering from, predisposed to, or at risk for various orthopedic-related diseases, disorders, or conditions. In other embodiments, sub-therapeutically effective amounts of first and second anti-connexin agents are used in the preparation of the article of manufacture that together will provide a desired therapeutic effect.

The invention includes a formulation comprising therapeutically effective amounts of a first anti-connexin agent and a second anti-connexin agent as described herein, for example, one or more anti-connexin polynucleotides and one or more anti-connexin peptides, peptidomimetics, or gap junction modifying agents in amounts effective to promote healing, improve recovery time, improve overall recovery outcome, decrease joint catracture, and/or decrease vascular damage during or following an orthopedic procedure or surgery. Such formulations include, for example, topical delivery forms and formulations, as well as formulations for injection, intstillation, and arthroscopic administration. Preferred formulations include, for example, a pharmaceutical composition of the invention which is formulated as a foam, spray or gel. In one embodiment, the gel is a polyoxyethylene-polyoxypropylene copolymer-based gel or a carboxymethylcellulose-based gel. In a preferred embodiment, the gel is a pluronic gel. In other embodiments, the invention provide a formulation comprising sub-therapeutically effective amounts of first and second anti-connexin agents that together will provide a desired therapeutic effect.

The invention includes methods for the use of therapeutically effective amounts of compositions comprising a first anti-connexin agent and a second anti-connexin agent as described herein, for example, one or more anti-connexin polynucleotides and one or more anti-connexin peptides, peptidomimetics, or gap junction modifying agents in the manufacture of a medicament for treating a subject during or following an orthopedic procedure or surgery or suffering from, predisposed to, or at risk for various orthopedic-related diseases, disorders, or conditions. Such medicaments include, for example, topical delivery forms and formulations, as well as formulations for injection, intstillation, and arthroscopic administration. Such medicaments include those for the treatment of a subject as disclosed herein. Such medicaments may optionally include reduced therapeutically effective amounts of a first anti-connexin agent and a second anti-connexin agent as described herein compared to amounts administered when such agents are not administered in combination, for example, reduced amounts of one or more anti-connexin polynucleotides and one or more anti-connexin peptides, peptidomimetics, or gap junction modifying agents, as noted herein. In other embodiments, sub-therapeutically effective amounts of anti-connexin agents are used that together will provide a desired therapeutic effect.

The invention includes methods of preparing a medicament for treating a subject during or following an orthopedic procedure or surgery or suffering from, predisposed to, or at risk for various orthopedic-related diseases, disorders, or conditions., comprising bringing together and an effective amount of an anti-connexin peptide (e.g., a hemichannel blocker), or a first anti-connexin agent and a second anti-connexin agent as described herein, including, for example, a first composition and a second composition wherein said first composition comprises an effective amount of an anti-connexin polynucleotide and said second composition comprises an effective amount of an anti-connexin peptide or peptidomimetic. Other embodiments preparing medicatments that include first and second compositions comprising therapeutically effective amounts of an anti-connexin polynucleotide, an anti-connexin peptide or peptidomimetic, a gap junction closing compound, a hemichannel closing compound, and/or a connexin carboxy-terminal polypeptide useful for treating a subject during or following an orthopedic procedure or surgery or suffering from, predisposed to or at risk of orthopedic diseases, disorders and/or condition. In other embodiments, sub-therapeutically effective amounts of anti-connexin agents to be used in combination are provided that together will provide a desired therapeutic effect.

The invention includes methods for the use of a therapeutically effective amount of a first anti-connexin agent and a second anti-connexin agent as described herein, for example, one or more anti-connexin polynucleotides and one or more anti-connexin peptides, peptidomimetics, or gap junction modifying agents in the manufacture of a dosage form useful for treating a subject during or following an orthopedic procedure or surgery or suffering from, predisposed to, or at risk for various orthopedic-related diseases, disorders, or conditions. Such dosage forms include, for example, topical delivery forms and formulations, as well as formulations for injection, intstillation, and arthroscopic administration. Such dosage forms include those for the treatment of a subject as disclosed herein. Such dosage forms preferably include the reduced therapeutically effective amounts of the one or more anti-connexin polynucleotides and one or more anti-connexin peptides, peptidomimetics, or gap junction modifying agents, as noted herein, including reduced amounts of a gap junction closing compound, a hemichannel closing compound, and/or a connexin carboxy-terminal polypeptide. In other embodiments, sub-therapeutically effective amounts of anti-connexin agents are used that together will provide a desired therapeutic effect.

In another aspect, the invention provides for the use of therapeutically effective amounts of a first anti-connexin agent and a second anti-connexin agent as described herein, for example, an anti-connexin polynucleotide (for example, anti-alpha-1 ODN) and an anti-connexin peptide or peptidomimetic, in the manufacture of a pharmaceutical product for the treatment of a subject during or following an orthopedic procedure or surgery or suffering from, predisposed to, or at risk for various orthopedic-related diseases, disorders, or conditions. In other embodiments, sub-therapeutically effective amounts of a first anti-connexin agent and a second anti-connexin agent as described herein are used to provide a desired therapeutic effect

In certain other aspects, the invention provides: a package comprising therapeutically effective amounts of one or more anti-connexin polynucleotides together with instructions for use in combination with one or more anti-connexin peptides, peptidomimetics, or gap junction modifying agents for treating a subject during or following an orthopedic procedure or surgery or suffering from, predisposed to, or at risk for various orthopedic-related diseases, disorders, or conditions. In other embodiments, the package contains sub-therapeutically effective amounts of one or more anti-connexin polynucleotides and one or more anti-connexin peptides, peptidomimetics, or gap junction modifying agents that, when used in combination provide are therapeutically effective.

These and other aspects of the present inventions, which are not limited to or by the information in this Brief Summary, are provided below.

### DETAILED DESCRIPTION

### Definitions

Subjects that may be treated using the compounds, compositions, and methods of treatment described herein include, for example, subjects suffering from, predisposed to or at risk of orthopedic diseases, disorders and/or conditions, and subjects who have undergone or will undergo an orthopedic surgical procedure.

As used herein, “subject” refers to any mammal, including humans, domestic and farm animals, and zoo, sports, or pet animals, such as dogs, horses, cats, sheep, pigs, cows, etc. The preferred mammal herein is a human, including adults, children, and the elderly.

As used herein, “preventing” means preventing in whole or in part, or ameliorating or controlling, or reducing, lessening, decreasing, retarding or halting the progress of a condition.

As used herein, an “effective amount” or “therapeutically effective amount” in reference to the compounds or compositions of the instant invention refers to the amount sufficient to induce a desired biological, pharmaceutical, or therapeutic result. That result can be alleviation of the signs, symptoms, or causes of a disease or disorder or condition, or any other desired alteration of a biological system. In the present invention, the result will involve the promotion of healing, preventing and/or decreasing pain, preventing and/or decreasing vascular damage, preventing and/or decreasing abnormal tissue formation inside and/or around a joint and/or preventing, decreasing or reversing of joint contracture in whole or in part, and/or improving recovery times, and/or improving overall recovery results, including increased post-operative mobility. The inventions described and claimed herein can also improve recovery times for post-orthopedic-surgical patients.

As used herein, the term “treating” refers to both therapeutic treatment and prophylactic or preventative measures.

As used herein, “anti-connexin agents” are compounds that affect or modulate the activity, expression or formation of a connexin, a connexin hemichannel (connexon), or a gap junction. Anti-connexin agents include, without limitation, antisense compounds (e.g. antisense polynucleotides), RNAi and siRNA compounds, antibodies and binding fragments thereof, and peptides and polypeptides, which include “peptidomimetics,” and peptide analogs. In addition to anti-connexin polynucleotides and anti-connexin peptides, peptidomimetics, or gap junction modifying agents, other anti-connexin agents include gap junction closing compounds (e.g., connexin phosphorylation compounds), hemichannel closing compounds useful for treating a subject during or following an orthopedic procedure or surgery or suffering from, predisposed to or at risk of orthopedic diseases, disorders and/or condition (e.g., connexin phosphorylation compounds), and connexin carboxy-terminal polypeptide (which can, e.g., block or disrupt ZO-1 protein interactions with connexins such as connexin 43) useful for treating a subject during or following an orthopedic procedure or surgery or suffering from, predisposed to or at risk of orthopedic diseases, disorders and/or condition. Preferred anti-connexin agents are anti-connexin 43 agents, anti-connexin 43 gap junction agents, and anti-connexin 43 hemichannel agents. Exemplary anti-connexin agents are discussed in further detail herein.

The terms “peptidomimetic” and “mimetic” include naturally occurring and synthetic chemical compounds that may have substantially the same structural and functional characteristics of protein regions which they mimic. In the case of connexins, these may mimic, for example, the extracellular loops of opposing connexins involved in connexin-connexin docking and cell-cell channel formation.

“Peptide analogs” refer to the compounds with properties analogous to those of the template peptide and may be non-peptide drugs. “Peptidomimetics” (also known as “mimetic peptides”), which include peptide-based compounds, also include such non-peptide based compounds such as peptide analogs. Peptidomimetics that are structurally similar to therapeutically useful peptides may be used to produce an equivalent or enhanced therapeutic or prophylactic effect. Generally, peptidomimetics are structurally identical or similar to a paradigm polypeptide (i.e., a polypeptide that has a biological or pharmacological function or activity), but can also have one or more peptide linkages optionally replaced by a linkage selected from the group consisting of, for example, —CH2NH—, —CH2S—, —CH2-CH2-, —CH═CH— (cis and trans), —COCH2-, —CH(OH)CH2-, and —CH2SO—. The mimetic can be either entirely composed of natural amino acids, or non-natural analogues of amino acids, or, is a chimeric molecule of partly natural peptide amino acids and partly non-natural analogs of amino acids. The mimetic can also comprise any amount of natural amino acid conservative substitutions as long as such substitutions also do not substantially alter mimetic activity. For example, a mimetic composition may be useful as an anti-connexin agent if it is capable of down-regulating biological actions or activities of connexins proteins or hemichannels, such as, for example, preventing the docking of hemichannels to form gap-junction-mediated cell-cell communications, or preventing the opening of hemichannels to expose the cell cytoplasm to the extracellular millieu.

Peptidomimetics, as well as gap junction modifying agents, including connexin phosphorylation compounds and connexin carboxy-terminal polypeptides, encompass those described or referenced herein, as well as those as may be known in the art, whether now known or later developed.

The terms “modulator” and “modulation” of connexin activity, as used herein in its various forms, refers to inhibition in whole or in part of the expression or action or activity of a connexin or connexin hemichannel or connexin gap junction and may function as anti-connexin agents.

In general, the term “protein” refers to any polymer of two or more individual amino acids (whether or not naturally occurring) linked via peptide bonds, as occur when the carboxyl carbon atom of the carboxylic acid group bonded to the alpha-carbon of one amino acid (or amino acid residue) becomes covalently bound to the amino nitrogen atom of the amino group bonded to the alpha-carbon of an adjacent amino acid. These peptide bond linkages, and the atoms comprising them (i.e., alpha-carbon atoms, carboxyl carbon atoms (and their substituent oxygen atoms), and amino nitrogen atoms (and their substituent hydrogen atoms)) form the “polypeptide backbone” of the protein. In addition, as used herein, the term “protein” is understood to include the terms “polypeptide” and “peptide” (which, at times, may be used interchangeably herein). Similarly, protein fragments, analogs, derivatives, and variants are may be referred to herein as “proteins,” and shall be deemed to be a “protein” unless otherwise indicated. The term “fragment” of a protein refers to a polypeptide comprising fewer than all of the amino acid residues of the protein. A “domain” of a protein is also a fragment, and comprises the amino acid residues of the protein often required to confer activity or function.

As used herein, “simultaneously” is used to mean that the one or more agents of the invention are administered concurrently, whereas the term “in combination” is used to mean they are administered, if not simultaneously or in physical combination, then “sequentially” within a timeframe that they both are available to act therapeutically. Thus, administration “sequentially” may permit one agent to be administered within minutes (for example, 1, 2, 3, 4, 5, 10, 15, 20, 25, 30) minutes or a matter of hours, days, weeks or months after the other provided that both the one or more anti-connexin polynucleotides and one or more anti-connexin peptides or peptidomimetics are concurrently present in effective amounts. The time delay between administration or administrations of the components will vary depending on the exact nature of the components, the interaction there between, and their respective half-lives.

Orthopedic diseases, disorders, and conditions are well known in the art and may include, for example, anterior cruciate ligament (acl) injury; arthritis of the shoulder; articular cartilage injury of the knee; bowlegs; broken back; broken hip (pelvis); broken leg; broken neck; bunions; bursitis; carpal tunnel syndrome; chronic low back pain; clubfoot; curvature of the spine (scoliosis); diabetic foot; dislocated elbow; dislocated hip; dupuytren's contracture; flatfoot; foot deformity; forearm fractures in children; hemophiliac arthritis; herniated disc (slipped disc); hip labral tear; hip arthritis; infectious arthritis; inflammatory hip conditions; intoeing; knee arthritis; knock knee; meniscal tear; osteoarthritis; osteonecrosis; osteoporosis; rheumatoid arthritis; rotator cuff injuries; scoliosis; shoulder arthritis; shoulder instability; shoulder pain; slipped disc (herniated disc); spinal stenosis/degenerative spondylolisthesis; spondylolisthesis; sports injuries to foot; sprained ankle; tendonitis; thumb arthritis; trigger finger, and joint contracture.

An orthopedic disease, disorder or condition also includes pain, vascular damage, swelling, inflammation, scarring and joint contractures which arise after an orthopedic injury. Representative examples of injuries which may give rise to inflammation, scarring and joint contractures include, for example, trauma (e.g., crushes, cuts, tears, disruptions, impacts, and tractions, especially in or around a joint), a fracture (which may occur in or around a joint, such as an elbow or hip), a subluxation, a dislocation (e.g., in the finger, elbow, shoulder, ankle, knee, or hip), or a joint (e.g., shoulder, elbow, hip, temporomandibular joint, facet, finger, knee, ankle, or toe) disruption and other bone, cartilage, tendon or ligament injuries, or there may be no identifiable cause (e.g., frozen shoulder).

Orthopedic surgical procedures are also well known in the art and include, for example, all procedures and/or therapeutic modalities presently known or later developed for treating a subject suffering from orthopedic related disease, disorders, or conditions, exemplary procedures may include; arthroscopy; artificial disc replacement; autologous chondrocyte implantation; bone graft; bunion removal; carpal tunnel release; clubfoot repair; corticosteroids (injection and medication); functional restoration program; hip arthroscopy; hip replacement; knee arthroscopy; minimally invasive total hip replacement; partial knee replacement; partial hip replacement; shoulder arthroscopy; shoulder joint replacement; total hip replacement; total knee replacement; wrist arthroscopy; knee arthroscopy and meniscectomy; shoulder arthroscopy and decompression; carpal tunnel release; knee arthroscopy and chondroplasty; removal of support implant; knee arthroscopy and anterior cruciate ligament reconstruction; knee replacement; repair of femoral neck fracture; repair of trochanteric fracture; débridement of skin/muscle/bone/fracture; knee arthroscopy repair of both menisci; hip replacement; shoulder arthroscopy/distal clavicle excision; repair of rotator cuff tendon; repair fracture of radius (bone)/ulna; laminectomy; repair of ankle fracture (bimalleolar type); shoulder arthroscopy and débridement; lumbar spinal fusion; repair fracture of the distal part of radius; low back intervertebral disc surgery; incise finger tendon sheath; repair of ankle fracture (fibula); repair of femoral shaft fracture; repair of trochanteric fracture as well as all procedures associated with general, specialized, or modified hand surgery; shoulder and elbow surgery; total joint reconstruction (arthroplasty); pediatric orthopedics; foot and ankle surgery; spine surgery; musculoskeletal oncology; surgical sports medicine; and orthopedic trauma.

### Anti-Connexin Agents

Anti-connexin agents of the invention described herein are capable of modulating or affecting the transport of molecules into and out of cells (e.g., blocking or inhibiting or downregulating). Thus, certain anti-connexin agents described herein modulate cellular communication (e.g., cell to cell). Certain anti-connexin agents are gap junction modulation agents. Certain anti-connexin agents modulate or effect transmission of molecules between the cell cytoplasm and the periplasmic or extracellular space. Such anti-connexin agents are generally targeted to connexins and/or connexin hemichannels (connexons) or to gap junctions themselves. Hemichannels and resulting gap junctions that comprise connexins are independently involved in the release or exchange of small molecules between the cell cytoplasm and an extracellular space or tissue in the case of open hemichannels, and between the cytoplasm of adjoining cell in the case of open gap junctions. Thus, an anti-connexin agents provided herein may directly or indirectly reduce coupling and communication between cells or reduce or block communication (or the transmission of molecules) between a cell and extracellular space or tissue, and the modulation of transport of molecules from a cell into an extracellular space or tissue (or from an extracellular space or tissue into a cell) or between adjoining cells is within the scope of anti-connexin agents and embodiments of the invention. Preferably, the connexin is connexin 43.

Any anti-connexin agent that is capable of eliciting a desired inhibition of the passage (e.g. transport) of molecules through a gap junction or connexin hemichannel may be used in embodiments of the invention. Any anti-connexin agents that modulates the passage of molecules through a gap junction or connexin hemichannel are also provided in particular embodiments (e.g., those that modulate, block or lessen the passage of molecules from the cytoplasm of a cell into an extracellular space or adjoining cell cytoplasm). Such anti-connexin agents may modulate the passage of molecules through a gap junction or connexin hemichannel with or without gap junction uncoupling (blocking the transport of molecules through gap junctions). Such compounds include, for example, proteins and polypeptides, polynucleotides, and other organic compounds, and they may, for example block the function or expression of a gap junction or a hemichannel in whole or in part, or downregulate the production of a connexin in whole or in part. Certain gap junction inhibitors are listed in Evans, W. H. and Boitano, S. Biochem. Soc. Trans. 29: 606-612 (2001). Other compounds include connexin phosphorylation compounds that close gap junctions and/or hemichannels, in whole or in part, and connexin carboxy-terminal polypeptides. Preferably, the connexin is connexin 43.

Certain anti-connexin agents provide downregulation of connexin expression (for example, by downregulation of mRNA transcription or translation) or otherwise decrease or inhibit the activity of a connexin protein, a connexin hemichannel or a gap junction. In the case of downregulation, this will have the effect of reducing direct cell-cell communication by gap junctions, or exposure of cell cytoplasm to the extracellular space by hemichannels, at the site at which connexin expression is downregulated. Anti-connexin 43 agents are preferred.

Examples of anti-connexin agents include agents that decrease or inhibit expression or function of connexin mRNA and/or protein or that decrease activity, expression or formation of a connexin, a connexin hemichannel or a gap junction. Anti-connexin agents include anti-connexin polynucleotides, such as antisense polynucleotides and other polynucleotides (such as polynucleotides having siRNA or ribozyme functionalities), as well as antibodies and binding fragments thereof, and peptides and polypeptides, including peptidomimetics and peptide analogs that modulate hemichannel or gap junction activity or function. Anti-connexin 43 agents are preferred.

Anti-Connexin Polynucleotides

Anti-connexin polynucleotides include connexin antisense polynucleotides as well as polynucleotides which have functionalities which enable them to downregulate connexin expression. Other suitable anti-connexin polynucleotides include RNAi polynucleotides and siRNA polynucleotides. Anti-connexin 43 polynucleotides are preferred.

Synthesis of antisense polynucleotides and other anti-connexin polynucleotides such as RNAi, siRNA, and ribozyme polynucleotides as well as polynucleotides having modified and mixed backbones is known to those of skill in the art. See e.g. Stein C. A. and Krieg A. M. (eds), Applied Antisense Oligonucleotide Technology, 1998 (Wiley-Liss). Methods of synthesizing antibodies and binding fragments as well as peptides and polypeptides, including peptidomimetics and peptide analogs are known to those of skill in the art. See e.g. Lihu Yang et al., Proc. Natl. Acad. Sci. U.S.A., 1; 95(18): 10836-10841 (September 1, 1998); Harlow and Lane (1988) “Antibodies: A Laboratory Manuel” Cold Spring Harbor Publications, New York; Harlow and Lane (1999) “Using Antibodies” A Laboratory Manuel, Cold Spring Harbor Publications, New York.

According to one aspect, the downregulation of connexin expression may be based generally upon the antisense approach using antisense polynucleotides (such as DNA or RNA polynucleotides), and more particularly upon the use of antisense oligodeoxynucleotides (ODN). These polynucleotides (e.g., ODN) target the connexin protein (s) to be downregulated. Typically the polynucleotides are single stranded, but may be double stranded.

The antisense polynucleotide may inhibit transcription and/or translation of a connexin. Preferably the polynucleotide is a specific inhibitor of transcription and/or translation from the connexin gene or mRNA, and does not inhibit transcription and/or translation from other genes or mRNAs. The product may bind to the connexin gene or mRNA either (i) 5′ to the coding sequence, and/or (ii) to the coding sequence, and/or (iii) 3′ to the coding sequence.

The antisense polynucleotide is generally antisense to a connexin mRNA, preferably connexin 43 mRNA. Such a polynucleotide may be capable of hybridizing to the connexin mRNA and may thus inhibit the expression of connexin by interfering with one or more aspects of connexin mRNA metabolism including transcription, mRNA processing, mRNA transport from the nucleus, translation or mRNA degradation. The antisense polynucleotide typically hybridizes to the connexin mRNA to form a duplex which can cause direct inhibition of translation and/or destabilization of the mRNA. Such a duplex may be susceptible to degradation by nucleases.

The antisense polynucleotide may hybridize to all or part of the connexin mRNA. Typically the antisense polynucleotide hybridizes to the ribosome binding region or the coding region of the connexin mRNA. The polynucleotide may be complementary to all of or a region of the connexin mRNA. For example, the polynucleotide may be the exact complement of all or a part of connexin mRNA. However, absolute complementarity is not required and polynucleotides which have sufficient complementarity to form a duplex having a melting temperature of greater than about 20° C., 30° C. or 40° C. under physiological conditions are particularly suitable for use in the present invention.

Thus the polynucleotide is typically a homologue of a sequence complementary to the mRNA. The polynucleotide may be a polynucleotide which hybridizes to the connexin mRNA under conditions of medium to high stringency such as 0.03M sodium chloride and 0.03M sodium citrate at from about 50° C. to about 60° C.

For certain aspects, suitable polynucleotides are typically from about 6 to 40 nucleotides in length. Preferably a polynucleotide may be from about 12 to about 35 nucleotides in length, or alternatively from about 12 to about 20 nucleotides in length or more preferably from about 18 to about 32 nucleotides in length. According to an alternative aspect, the polynucleotide may be at least about 40, for example at least about 60 or at least about 80, nucleotides in length and up to about 100, about 200, about 300, about 400, about 500, about 1000, about 2000 or about 3000 or more nucleotides in length.

The connexin protein or proteins targeted by the polynucleotide will be dependent upon the site at which downregulation is to be effected. This reflects the non-uniform make-up of gap junction(s) at different sites throughout the body in terms of connexin sub-unit composition. The connexin is a connexin that naturally occurs in a human or animal in one aspect or naturally occurs in the tissue in which connexin expression or activity is to be decreased. The connexin gene (including coding sequence) generally has homology with the coding sequence of one or more of the specific connexins mentioned herein, such as homology with the connexin 43 coding sequence shown in Table 8. The connexin is typically an α or β connexin. Preferably the connexin is an a connexin and is expressed in the tissue to be treated.

Some connexin proteins are however more ubiquitous than others in terms of distribution in tissue. One of the most widespread is connexin 43. Polynucleotides targeted to connexin 43 are particularly suitable for use in the present invention. In other aspects other connexins are targeted.

Anti-connexin polynucleotides include connexin antisense polynucleotides as well as polynucleotides which have functionalities which enable them to downregulate connexin expression. Other suitable anti-connexin polynucleotides include RNAi polynucleotides and SiRNA polynucleotides.

In one preferred aspect, the antisense polynucleotides are targeted to the mRNA of one connexin protein only. Most preferably, this connexin protein is connexin 43. In another aspect, connexin protein is connexin 26, 30, 31.1, 32, 36, 37, 40, or 45. In other aspects, the connexin protein is connexin 30.3, 31, 40.1, or 46.6.

It is also contemplated that polynucleotides targeted to separate connexin proteins be used in combination (for example 1, 2, 3, 4 or more different connexins may be targeted). For example, polynucleotides targeted to connexin 43, and one or more other members of the connexin family (such as connexin 26, 30, 30.3, 31.1, 32, 36, 37, 40, 40.1, 45, and 46.6) can be used in combination.

Alternatively, the antisense polynucleotides may be part of compositions which may comprise polynucleotides to more than one connexin protein. Preferably, one of the connexin proteins to which polynucleotides are directed is connexin 43. Other connexin proteins to which oligodeoxynucleotides are directed may include, for example, connexins 26, 30, 30.3, 31.1, 32, 36, 37, 40, 40.1, 45, and 46.6. Suitable exemplary polynucleotides (and ODNs) directed to various connexins are set forth in Table 1.

Individual antisense polynucleotides may be specific to a particular connexin, or may target 1, 2, 3 or more different connexins. Specific polynucleotides will generally target sequences in the connexin gene or mRNA which are not conserved between connexins, whereas non-specific polynucleotides will target conserved sequences for various connexins.

The polynucleotides for use in the invention may suitably be unmodified phosphodiester oligomers. Such oligodeoxynucleotides may vary in length. A 30 mer polynucleotide has been found to be particularly suitable.

Many aspects of the invention are described with reference to oligodeoxynucleotides. However it is understood that other suitable polynucleotides (such as RNA polynucleotides) may be used in these aspects.

The antisense polynucleotides may be chemically modified. This may enhance their resistance to nucleases and may enhance their ability to enter cells. For example, phosphorothioate oligonucleotides may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-O-methylribonucleotide methylphosphonates. Alternatively mixed backbone oligonucleotides (“MBOs”) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides. Methods of preparing modified backbone and mixed backbone oligonucleotides are known in the art.

The precise sequence of the antisense polynucleotide used in the invention will depend upon the target connexin protein. In one embodiment, suitable connexin antisense polynucleotides can include polynucleotides such as oligodeoxynucleotides selected from the following sequences set forth in Table 1:

TABLE 1

| | | |
|---|---|---|
| 5′ GTA ATT GCG GCA AGA AGA ATT GTT TCT GTC 3′ | (connexin 43) | (SEQ. ID. NO: 1) |
| 5′ GTA ATT GCG GCA GGA GGA ATT GTT TCT GTC 3′ | (connexin 43) | (SEQ. ID. NO: 2) |
| 5′ GGC AAG AGA CAC CAA AGA CAC TAC CAG CAT 3′ | (connexin 43) | (SEQ. ID. NO: 3) |
| 5′ TCC TGA GCA ATA CCT AAC GAA CAA ATA 3′ | (connexin 26) | (SEQ. ID. NO: 4) |
| 5′ CAT CTC CTT GGT GCT CAA CC 3′ | (connexin 37) | (SEQ. ID. NO: 5) |
| 5′ CTG AAG TCG ACT TGG CTT GG 3′ | (connexin 37) | (SEQ. ID. NO: 6) |
| 5′ CTC AGA TAG TGG CCA GAA TGC 3′ | (connexin 30) | (SEQ. ID. NO: 7) |
| 5′ TTG TCC AGG TGA CTC CAA GG 3′ | (connexin 30) | (SEQ. ID. NO: 8) |
| 5′ CGT CCG AGC CCA GAA AGA TGA GGT C 3′ | (connexin 31.1) | (SEQ. ID. NO: 9) |
| 5′ AGA GGC GCA CGT GAG ACA C 3′ | (connexin 31.1) | (SEQ. ID. NO: 10) |
| 5′ TGA AGA CAA TGA AGA TGT T 3′ | (connexin 31.1) | (SEQ. ID. NO: 11) |
| 5′ TTT CTT TTC TAT GTG CTG TTG GTG A 3′ | (connexin 32) | (SEQ. ID. NO: 12) |

Suitable polynucleotides for the preparation of the combined polynucleotide compositions described herein include for example, polynucleotides to Connexin Cx43 and polynucleotides for connexins 26, 30, 31.1, 32 and 37 as described in Table 1 above.

Although the precise sequence of the antisense polynucleotide used in the invention will depend upon the target connexin protein, for connexin 43, antisense polynucleotides having the following sequences have been found to be particularly suitable:

(SEQ. ID. NO: 1)
GTA ATT GCG GCA AGA AGA ATT GTT TCT GTC;
(SEQ. ID. NO: 2)
GTA ATT GCG GCA GGA GGA ATT GTT TCT GTC;
and
(SEQ. ID. NO: 3)
GGC AAG AGA CAC CAA AGA CAC TAC CAG CAT.

For example, suitable antisense polynucleotides for connexins 26, 31.1 and 32 have the following sequences:

(SEQ. ID. NO: 4)
5′ TCC TGA GCA ATA CCT AAC GAA CAA ATA
(connexin 26);
(SEQ. ID. NO: 9)
5′ CGT CCG AGC CCA GAA AGA TGA GGT C
(connexin 31.1);
and
(SEQ. ID. NO: 12)
5′ TTT CTT TTC TAT GTG CTG TTG GTG A
(connexin 32).

Other connexin antisense polynucleotide sequences useful according to the methods of the present invention include:

(SEQ. ID. NO: 5)
5′ CAT CTC CTT GGT GCT CAA CC 3′ (connexin 37);
(SEQ. ID. NO: 6)
5′ CTG AAG TCG ACT TGG CTT GG 3′ (connexin 37);
(SEQ. ID. NO: 7)
5′ CTC AGA TAG TGG CCA GAA TGC 3′ (connexin 30);
(SEQ. ID. NO: 8)
5′ TTG TCC AGG TGA CTC CAA GG 3′ (connexin 30);
(SEQ. ID. NO: 10)
5′ AGA GGC GCA CGT GAG ACA C 3′ (connexin 31.1);
and
(SEQ. ID. NO: 11)
5′ TGA AGA CAA TGA AGA TGT T 3′ (connexin 31.1).

Polynucleotides, including ODN's, directed to connexin proteins can be selected in terms of their nucleotide sequence by any convenient, and conventional, approach. For example, the computer programs MacVector and OligoTech (from Oligos etc. Eugene, Oreg., USA) can be used. Once selected, the ODN's can be synthesized using a DNA synthesizer.

Polynucleotide Homologues

Homology and homologues are discussed herein (for example, the polynucleotide may be a homologue of a complement to a sequence in connexin mRNA). Such a polynucleotide typically has at least about 70% homology, preferably at least about 80%, at least about 90%, at least about 95%, at least about 97% or at least about 99% homology with the relevant sequence, for example over a region of at least about 15, at least about 20, at least about 40, at least about 100 more contiguous nucleotides (of the homologous sequence).

Homology may be calculated based on any method in the art. For example the UWGCG Package provides the BESTFIT program which can be used to calculate homology (for example used on its default settings) (Devereux et al (1984) Nucleic Acids Research 12, p 387-395). The PILEUP and BLAST algorithms can be used to calculate homology or line up sequences (typically on their default settings), for example as described in Altschul S. F. (1993) J Mol Evol 36: 290-300; Altschul, S, F et al (1990) J Mol Biol 215: 403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

The BLAST algorithm parameters W, T and X determine the sensitivity and speed of the alignment. The BLAST program uses as defaults a word length (W), the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to a second sequence is less than about 1, preferably less than about 0.1, more preferably less than about 0.01, and most preferably less than about 0.001.

The homologous sequence typically differs from the relevant sequence by at least about (or by no more than about) 2, 5, 10, 15, 20 more mutations (which may be substitutions, deletions or insertions). These mutations may be measured across any of the regions mentioned above in relation to calculating homology.

The homologous sequence typically hybridizes selectively to the original sequence at a level significantly above background. Selective hybridization is typically achieved using conditions of medium to high stringency (for example 0.03M sodium chloride and 0.03M sodium citrate at from about 50° C. to about 60° C.). However, such hybridization may be carried out under any suitable conditions known in the art (see Sambrook et al. (1989), Molecular Cloning: A Laboratory Manual). For example, if high stringency is required, suitable conditions include 0.2×SSC at 60° C. If lower stringency is required, suitable conditions include 2×SSC at 60° C.

Peptide and Polypeptide Anti-Connexin Agents

Binding proteins, including peptides, peptidomimetics, antibodies, antibody fragments, and the like, are also suitable modulators of gap junctions and hemichannels.

Binding proteins include, for example, monoclonal antibodies, polyclonal antibodies, antibody fragments (including, for example, Fab, F(ab′)₂ and Fv fragments; single chain antibodies; single chain Fvs; and single chain binding molecules such as those comprising, for example, a binding domain, hinge, CH2 and CH3 domains, recombinant antibodies and antibody fragments which are capable of binding an antigenic determinant (i.e., that portion of a molecule, generally referred to as an epitope) that makes contact with a particular antibody or other binding molecule. These binding proteins, including antibodies, antibody fragments, and so on, may be chimeric or humanized or otherwise made to be less immunogenic in the subject to whom they are to be administered, and may be synthesized, produced recombinantly, or produced in expression libraries. Any binding molecule known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g. connexin, hemichannel, or associated molecules).

Binding molecules will generally have a desired specificity, including but not limited to binding specificity, and desired affinity. Affinity, for example, may be a Kₐ of greater than or equal to about 10⁴ M⁻¹, greater than or equal to about 10⁶ M⁻¹, greater than or equal to about 10⁷ M⁻¹, greater than or equal to about 10⁸ M⁻¹. Affinities of even greater than about 10⁸ M⁻¹ are suitable, such as affinities equal to or greater than about 10⁹ M⁻¹, about 10¹⁰ M⁻¹, about 10¹¹ M⁻¹, and about 10¹² M⁻¹. Affinities of binding proteins according to the present invention can be readily determined using conventional techniques, for example those described by Scatchard et al., 1949 Ann. N.Y. Acad. Sci. 51: 660.

By using data obtained from hydropathy plots, it has been proposed that a connexin contains four-transmembrane-spanning regions and two short extra-cellular loops. The positioning of the first and second extracellular regions of connexin was further characterized by the reported production of anti-peptide antibodies used for immunolocalization of the corresponding epitopes on split gap junctions. Goodenough D. A. J Cell Biol 107: 1817-1824 (1988); Meyer R. A., J Cell Biol 119: 179-189 (1992).

The extracellular domains of a hemichannel contributed by two adjacent cells “dock” with each other to form complete gap junction channels. Reagents that interfere with the interactions of these extracellular domains can impair cell-to-cell communication. Peptide inhibitors of gap junctions and hemichannels have been reported. See for example Berthoud, V. M. et al., Am J. Physiol. Lung Cell Mol. Physiol. 279: L619-L622 (2000); Evans, W. H. and Boitano, S. Biochem. Soc. Trans. 29: 606-612, and De Vriese A. S., et al. Kidney Int. 61: 177-185 (2001). Short peptides corresponding to sequences within the extracellular loops of connexins were said to inhibit intercellular communication. Boitano S. and Evans W. Am J Physiol Lung Cell Mol Physiol 279: L623-L630 (2000). The use of peptides as inhibitors of cell-cell channel formation produced by connexin (Cx) 32 expressed in paired Xenopus oocytes has also been reported. Dahl G, et al., Biophys J 67: 1816-1822 (1994). Berthoud, V. M. and Seul, K. H., summarized some of these results. Am J., Physiol. Lung Cell Mol. Physiol. 279: L619-L622 (2000).

Anti-connexin agents include peptides comprising an amino acid sequence corresponding to a transmembrane region (e.g. 1^{st} to 4^{th}) of a connexin (e.g. connexin 45, 43, 26, 30, 31.1, and 37). Anti-connexin agents may comprise a peptide comprising an amino acid sequence corresponding to a portion of a transmembrane region of a connexin 45. Anti-connexin agents include a peptide having an amino acid sequence that comprises about 5 to 20 contiguous amino acids of SEQ.ID.NO:13, a peptide having an amino acid sequence that comprises about 8 to 15 contiguous amino acids of SEQ.ID.NO:13, or a peptide having an amino acid sequence that comprises about 11 to 13 contiguous amino acids of SEQ.ID.NO:13. Other embodiments are directed to an anti-connexin agent that is a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ.ID.NO:13. In certain anti-connexin agents provided herein, the extracellular domains of connexin 45 corresponding to the amino acids at positions 46-75 and 199-228 of SEQ.ID.NO:13 may be used to develop the particular peptide sequences. Certain peptides described herein have an amino acid sequence corresponding to the regions at positions 46-75 and 199-228 of SEQ.ID.NO: 13. The peptides need not have an amino acid sequence identical to those portions of SEQ.ID.NO:13, and conservative amino acid changes may be made such that the peptides retain binding activity or functional activity. Alternatively, the peptide may target regions of the connexin protein other than the extracellular domains (e.g. the portions of SEQ.ID.NO:13 not corresponding to positions 46-75 and 199-228).

Also, suitable anti-connexin agents comprise a peptide comprising an amino acid sequence corresponding to a portion of a transmembrane region of a connexin 43. Anti-connexin agents include peptides having an amino acid sequence that comprises about 5 to 20 contiguous amino acids of SEQ.ID.NO:14, peptides having an amino acid sequence that comprises about 8 to 15 contiguous amino acids of SEQ.ID.NO:14, or peptides having an amino acid sequence that comprises about 11 to 13 contiguous amino acids of SEQ.ID.NO:14. Other anti-connexin agents include a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ.ID.NO:14. Other anti-connexin agents comprise the extracellular domains of connexin 43 corresponding to the amino acids at positions 37-76 and 178-208 of SEQ.ID.NO:14. Anti-connexin agents include peptides described herein which have an amino acid sequence corresponding to the regions at positions 37-76 and 178-208 of SEQ.ID.NO:14. The peptides need not have an amino acid sequence identical to those portions of SEQ.ID.NO:14, and conservative amino acid changes may be made such that the peptides retain binding activity or functional activity. Alternatively, peptides may target regions of the connexin protein other than the extracellular domains (e.g. the portions of SEQ.ID.NO:14 not corresponding to positions 37-76 and 178-208).

```
Connexin 45
(SEQ ID NO. 13)
Met Ser Trp Ser Phe Leu Thr Arg Leu Leu Glu Glu Ile His Asn His
1 5 10 15
Ser Thr Phe Val Gly Lys Ile Trp Leu Thr Val Leu Ile Val Phe Arg
                        20 25 30
Ile Val Leu Thr Ala Val Gly Gly Glu Ser Ile Tyr Tyr Asp Glu Gln
                35 40 45
Ser Lys Phe Val Cys Asn Thr Glu Gln Pro Gly Cys Glu Asn Val Cys
        50 55 60
Tyr Asp Ala Phe Ala Pro Leu Ser His Val Arg Phe Trp Val Phe Gln
65 70 75 80
Ile Ile Leu Val Ala Thr Pro Ser Val Met Tyr Leu Gly Tyr Ala Ile
                                85 90 95
His Lys Ile Ala Lys Met Glu His Gly Glu Ala Asp Lys Lys Ala Ala
                        100 105 110
Arg Ser Lys Pro Tyr Ala Met Arg Trp Lys Gln His Arg Ala Leu Glu
                115 120 125
Glu Thr Glu Glu Asp Asn Glu Glu Asp Pro Met Met Tyr Pro Glu Met
        130 135 140
Glu Leu Glu Ser Asp Lys Glu Asn Lys Glu Gln Ser Gln Pro Lys Pro
145 150 155 160
Lys His Asp Gly Arg Arg Arg Ile Arg Glu Asp Gly Leu Met Lys Ile
                                165 170 175
Tyr Val Leu Gln Leu Leu Ala Arg Thr Val Phe Glu Val Gly Phe Leu
                        180 185 190
Ile Gly Gln Tyr Phe Leu Tyr Gly Phe Gln Val His Pro Phe Tyr Val
                195 200 205
Cys Ser Arg Leu Pro Cys Pro His Lys Ile Asp Cys Phe Ile Ser Arg
        210 215 220
Pro Thr Glu Lys Thr Ile Phe Leu Leu Ile Met Tyr Gly Val Thr Gly
225 230 235 240
Leu Cys Leu Leu Leu Asn Ile Trp Glu Met Leu His Leu Gly Phe Gly
                                245 250 255
Thr Ile Arg Asp Ser Leu Asn Ser Lys Arg Arg Glu Leu Glu Asp Pro
                        260 265 270
Gly Ala Tyr Asn Tyr Pro Phe Thr Trp Asn Thr Pro Ser Ala Pro Pro
                275 280 285
Gly Tyr Asn Ile Ala Val Lys Pro Asp Gln Ile Gln Tyr Thr Glu Leu
        290 295 300
Ser Asn Ala Lys Ile Ala Tyr Lys Gln Asn Lys Ala Asn Thr Ala Gln
305 310 315 320
Glu Gln Gln Tyr Gly Ser His Glu Glu Asn Leu Pro Ala Asp Leu Glu
                                325 330 335
Ala Leu Gln Arg Glu Ile Arg Met Ala Gln Glu Arg Leu Asp Leu Ala
                        340 345 350
Val Gln Ala Tyr Ser His Gln Asn Asn Pro His Gly Pro Arg Glu Lys
                355 360 365
Lys Ala Lys Val Gly Ser Lys Ala Gly Ser Asn Lys Ser Thr Ala Ser
        370 375 380
Ser Lys Ser Gly Asp Gly Lys Asn Ser Val Trp Ile
385 390 395
Connexin 43
(SEQ ID NO. 14)
Met Gly Asp Trp Ser Ala Leu Gly Lys Leu Leu Asp Lys Val Gln Ala
1 5 10 15
Tyr Ser Thr Ala Gly Gly Lys Val Trp Leu Ser Val Leu Phe Ile Phe
                        20 25 30
Arg Ile Leu Leu Leu Gly Thr Ala Val Glu Ser Ala Trp Gly Asp Glu
                35 40 45
Gln Ser Ala Phe Arg Cys Asn Thr Gln Gln Pro Gly Cys Glu Asn Val
        50 55 60
Cys Tyr Asp Lys Ser Phe Pro Ile Ser His Val Arg Phe Trp Val Leu
65 70 75 80
Gln Ile Ile Phe Val Ser Val Pro Thr Leu Leu Tyr Leu Ala His Val
                                85 90 95
Phe Tyr Val Met Arg Lys Glu Glu Lys Leu Asn Lys Lys Glu Glu Glu
                        100 105 110
Leu Lys Val Ala Gln Thr Asp Gly Val Asn Val Asp Met His Leu Lys
                115 120 125
Gln Ile Glu Ile Lys Lys Phe Lys Tyr Gly Ile Glu Glu His Gly Lys
        130 135 140
Val Lys Met Arg Gly Gly Leu Leu Arg Thr Tyr Ile Ile Ser Ile Leu
145 150 155 160
Phe Lys Ser Ile Phe Glu Val Ala Phe Leu Leu Ile Gln Trp Tyr Ile
                                165 170 175
Tyr Gly Phe Ser Leu Ser Ala Val Tyr Thr Cys Lys Arg Asp Pro Cys
                        180 185 190
Pro His Gln Val Asp Cys Phe Leu Ser Arg Pro Thr Glu Lys Thr Ile
                195 200 205
Phe Ile Ile Phe Met Leu Val Val Ser Leu Val Ser Leu Ala Leu Asn
        210 215 220
Ile Ile Glu Leu Phe Tyr Val Phe Phe Lys Gly Val Lys Asp Arg Val
225 230 235 240
Lys Gly Lys Ser Asp Pro Tyr His Ala Thr Ser Gly Ala Leu Ser Pro
                                245 250 255
Ala Lys Asp Cys Gly Ser Gln Lys Tyr Ala Tyr Phe Asn Gly Cys Ser
                        260 265 270
Ser Pro Thr Ala Pro Leu Ser Pro Met Ser Pro Pro Gly Tyr Lys Leu
                275 280 285
Val Thr Gly Asp Arg Asn Asn Ser Ser Cys Arg Asn Tyr Asn Lys Gln
        290 295 300
Ala Ser Glu Gln Asn Trp Ala Asn Tyr Ser Ala Glu Gln Asn Arg Met
305 310 315 320
Gly Gln Ala Gly Ser Thr Ile Ser Asn Ser His Ala Gln Pro Phe Asp
                                325 330 335
Phe Pro Asp Asp Asn Gln Asn Ser Lys Lys Leu Ala Ala Gly His Glu
                        340 345 350
Leu Gln Pro Leu Ala Ile Val Asp Gln Arg Pro Ser Ser Arg Ala Ser
                355 360 365
Ser Arg Ala Ser Ser Arg Pro Arg Pro Asp Asp Leu Glu Ile
        370 375 380
```

The anti-connexin peptides may comprise sequences corresponding to a portion of the connexin extracellular domains with conservative amino acid substitutions such that peptides are functionally active anti-connexin agents. Exemplary conservative amino acid substitutions include for example the substitution of a nonpolar amino acid with another nonpolar amino acid, the substitution of an aromatic amino acid with another aromatic amino acid, the substitution of an aliphatic amino acid with another aliphatic amino acid, the substitution of a polar amino acid with another polar amino acid, the substitution of an acidic amino acid with another acidic amino acid, the substitution of a basic amino acid with another basic amino acid, and the substitution of an ionizable amino acid with another ionizable amino acid.

Exemplary peptides targeted to connexin 43 are shown below in Table 2. M1, 2, 3 and 4 refer to the 1^{st} to 4^{th} transmembrane regions of the connexin 43 protein respectively. E1 and E2 refer to the first and second extracellular loops respectively.

TABLE 2
Peptidic Inhibitors of Intercellular
Communication (cx43)

| | | |
|---|---|---|
| FEVAFLLIQWI | M3 & E2 | (SEQ. ID. NO: 15) |
| LLIQWYIGFSL | E2 | (SEQ. ID. NO: 16) |
| SLSAVYTCKRDPCPHQ | E2 | (SEQ. ID. NO: 17) |
| VDCFLSRPTEKT | E2 | (SEQ. ID. NO: 18) |
| SRPTEKTIFII | E2 & M4 | (SEQ. ID. NO: 19) |
| LGTAVESAWGDEQ | M1 & E1 | (SEQ. ID. NO: 20) |
| QSAFRCNTQQPG | E1 | (SEQ. ID. NO: 21) |
| QQPGCENVCYDK | E1 | (SEQ. ID. NO: 22) |
| VCYDKSFPISHVR | El | (SEQ. ID. NO: 23) |

Table 3 provides additional exemplary connexin peptides used in inhibiting hemichannel or gap junction function. In other embodiments, conservative amino acid changes are made to the peptides or fragments thereof.

TABLE 3
Additional Peptidic Inhibitors of Intercellular
Communication (cx32, cx43)

| | | | AA's and |
|---|---|---|---|
| Connexin | Location | | Sequence |
| Cx32 | E1 39-77 | AAESVWGDEIKSSFICNTLQPGCNS | (SEQ. ID. NO: 24) |
| | | VCYDHFFPISHVR | |
| Cx32 | E1 41-52 | ESVWGDEKSSFI | (SEQ. ID. NO: 25) |
| Cx32 | E1 52-63 | ICNTLQPGCNSV | (SEQ. ID. NO: 26) |
| Cx32 | E1 62-73 | SVCYDHFFPISH | (SEQ. ID. NO: 27) |
| Cx32 | E2 64-188 | RLVKCEAFPCPNTVDCFVSRPTEKT | (SEQ. ID. NO: 28) |
| Cx32 | E2 166-177 | VKCEAFPCPNTV | (SEQ. ID. NO: 29) |
| Cx32 | E2 177-188 | VDCFVSRPTEKT | (SEQ. ID. NO: 30) |
| Cx32 | E1 63-75 | VCYDHFFPISHVR | (SEQ. ID. NO: 31) |
| Cx32 | E1 45-59 | VWGDEKSSFICNTLQPGY | (SEQ. ID. NO: 32) |
| Cx32 | E1 46-59 | DEKSSFICNTLQPGY | (SEQ. ID. NO: 33) |
| Cx32 | E2 182-192 | SRPTEKTVFTV | (SEQ. ID. NO: 34) |
| Cx32/Cx43 | E2 182-188/ | SRPTEKT | (SEQ. ID. NO: 35) |
| | 201-207 | | |
| Cx32 | E1 52-63 | ICNTLQPGCNSV | (SEQ. ID. NO: 36) |
| Cx40 | E2 177-192 | FLDTLHVCRRSPCPHP | (SEQ. ID. NO: 37) |
| Cx43 | E2 188-205 | KRDPCHQVDCFLSRPTEK | (SEQ. ID. NO: 38) |

Table 4 provides the extracellular loops for connexin family members which are used to develop peptide inhibitors for use as described herein. The peptides and provided in Table 4, and fragments thereof, are used as peptide inhibitors in certain non-limiting embodiments. In other non-limiting embodiments, peptides comprising from about 8 to about 15, or from about 11 to about 13 amino contiguous amino acids of the peptides in this Table 4 are peptide inhibitors. Conservative amino acid changes may be made to the peptides or fragments thereof.

TABLE 4
Extracellular loops for various connexin family members
E1

| | | |
|---|---|---|
| huCx26 | KEVWGDEQADFVCNTLQPGCKNVCYDHYFPISHIR | (SEQ. ID. NO: 39) |
| huCx30 | QEVWGDEQEDFVCNTLQPGCKNVCYDHFFPVSHIR | (SEQ. ID. NO: 40) |
| huCx30.3 | EEVWDDEQKDFVCNTKQPGCPNVCYDEFFPVSHVR | (SEQ. ID. NO: 41) |
| huCx31 | ERVWGDEQKDFDCNTKQPGCTNVCYDNYFPISNIR | (SEQ. ID. NO: 42) |
| huCx31.1 | ERVWSDDHKDFDCNTRQPGCSNVCFDEFFPVSHVR | (SEQ. ID. NO: 43) |
| huCx32 | ESVWGDEKSSFICNTLQPGCNSVCYDQFFPISHVR | (SEQ. ID. NO: 44) |
| huCx36 | ESVWGDEQSDFECNTAQPGCTNVCYDQAFPISHIR | (SEQ. ID. NO: 45) |
| huCx37 | ESVWGDEQSDFECNTAQPGCTNVCYDQAFPISHIR | (SEQ. ID. NO: 46) |
| huCx40.1 | RPVYQDEQERFVCNTLQPGCANVCYDVFSPVSHLR | (SEQ. ID. NO: 47) |
| huCx43 | ESAWGDEQSAFRCNTQQPGCENVCYDKSFPISHVR | (SEQ. ID. NO: 48) |
| huCx46 | EDVWGDEQSDFTCNTQQPGCBNVCYBRAFPISHIR | (SEQ. ID. NO: 49) |
| huCx46.6 | EAIYSDEQAKFTCNTRQPGCDNVCYDAFAPLSHVR | (SEQ. ID. NO: 50) |
| huCx40 | ESSWGDEQADFRCDTIQPGCQNVCTDQAFPISHIR | (SEQ. ID. NO: 51) |
| huCx45 | GESIYYDEQSKFVCNTEQPGCENVCYDAFAPLSHVR | (SEQ. ID. NO: 52) |

E2

| | | |
|---|---|---|
| huCx26 | MYVFYVMYDGFSMQRLVKCNAWPCPNTVDCFVSRPTEKT | (SEQ. ID. NO: 53) |
| huCx30 | MYVFYFLYNGYHLPWVLKCGIDPCPNLVDCFISRPTEKT | (SEQ. ID. NO: 54) |
| huCx30.3 | LYIFHRLYKDYDMPRVVACSVEPCPHTVDCYISRPTEKK | (SEQ. ID. NO: 55) |
| huCx31 | LYLLHTLWHGFNMPRLVQCANVAPCPNIVDCYIARPTEKK | (SEQ. ID. NO: 56) |
| huCx31.1 | LYVFHSFYPKYILPPVVKCHADPCPNIVDCFISKPSEKN | (SEQ. ID. NO: 57) |
| huCx32 | MYVFYLLYPGYAMVRLVKCDVYPCPNTVDCFVSRPTEKT | SEQ. ID. NO: 58) |
| huCx36 | LYGWTMEPVFVCQRAPCPYLVDCFVSRPTEKT | (SEQ. ID. NO: 59) |
| huCx37 | LYGWTMEPVFVCQRAPCPYLVDCFVSRPTEKT | (SEQ. ID. NO: 60) |
| huCx40.1 | GALHYFLFGFLAPKKFPCTRPPCTGVVDCYVSRPTSKS | (SEQ. ID. NO: 61) |
| huCx43 | LLIQWYIYGFSLSAVYTCKRDPCPHQVDCFLSRPTEKT | (SEQ. ID. NO: 62) |
| huCx46 | IAGQYFLYGFELKPLYRCDRWPCPNTVDCFISRPTEKT | (SEQ. ID. NO: 63) |
| huCx46.6 | LVGQYLLYGFEVRPFFPCSRQPCPHVVDCFVSRPTEKT | (SEQ. ID. NO: 64) |
| huCx40 | IVGQYFIYGIFLTTLHVCRRSPCPHPVNCYVSRPTEKN | (SEQ. ID. NO: 65) |
| huCx45 | LIGQYFLYGFQVHPFYVCSRLPCHPKIDCFISRPTEKT | (SEQ. ID. NO: 66) |

Table 5 provides the extracellular domain for connexin family members which may be used to develop peptide anti-connexin agents. The peptides and provided in Table 5, and fragments thereof, may also be used as peptide anti-connexin agents. Such peptides may comprise from about 8 to about 15, or from about 11 to about 13 amino contiguous amino acids of the peptide sequence in this Table 5. Conservative amino acid changes may be made to the peptides or fragments thereof.

TABLE 5
Extracellular domains

| | | |
|---|---|---|
| Peptide | VDCFLSRPTEKT | (SEQ. ID. NO: 18) |
| Peptide | SRPTEKTIFII | (SEQ. ID. NO: 19) |
| huCx43 | LLIQWYIYGFSLSAVYTCKRDPCPHQVDCFLSRPTEKTIFII | (SEQ. ID. NO: 67) |
| huCx26 | MYVFYVMYDGFSMQRLVKCNAWPCPNTVDCFVSRPTEKTVFTV | (SEQ. ID. NO: 68) |
| huCx30 | YVFYFLYNGYHLPWVLKCGIDPCPNLVDCFISRPTEKTVFTI | (SEQ. ID. NO: 69) |
| huCx30.3 | LYIFHRLYKDYDMPRVVACSVEPCPHTVDCYISRPTEKKVFTY | (SEQ. ID. NO: 70) |
| huCx31 | LYLLHTLWHGFNMPRLVQCANVAPCPNIVDCYIARPTEKKTY | (SEQ. ID. NO: 71) |
| huCx31.1 | LYVFHSFYPKYILPPVVKCHADPCPNIVDCFISKPSEKNIFTL | (SEQ. ID. NO: 72) |
| huCx32 | MYVFYLLYPGYAMVRLVKCDVYPCPNTVDCFVSRPTEKTVFTV | (SEQ. ID. NO: 73) |
| huCx36 | LYGWTMEPVFVCQRAPCPYLVDCFVSRPTEKTIFII | (SEQ. ID. NO: 74) |
| huCx37 | LYGWTMEPVFVCQRAPCPYLVDCFVSRPTEKTIFII | (SEQ. ID. NO: 75) |
| huCx40.1 | GALHYFLFGFLAPKKFPCTRPPCTGVVDCYVSRPTEKSLLML | (SEQ. ID. NO: 76) |
| huCx46 | IAGQYFLYGFELKPLYRCDRWPCPNTVDCFISRPTEKTIFII | (SEQ. ID. NO: 77) |
| huCx46.6 | LVGQYLLYGFEVRPFFPCSRQPCPHVVDCFVSRPTEKTVFLL | (SEQ. ID. NO: 78) |
| huCx40 | IVGQYFIYGIFLTTLHVCRRSPCPHPVNCYSRPTEKNVFIV | (SEQ. ID. NO: 79) |
| huCx45 | LIGQYFLYGFQVHPFYVCSRLPCHPKIDCFISRPTEKTIFLL | (SEQ. ID. NO: 80) |

Table 6 provides peptides inhibitors of connexin 40 shown with reference to the extracellular loops (E1 and E2) of connexin 40. The bold amino acids are directed to the transmembrane regions of connexin 40.

TABLE 6
Cx40 peptide inhibitors
E2

| | |
|---|---|
| LGTAAESSWGDEQADFRCDTIQPGCQNVCTDQAFPISHIRFWVLQ | (SEQ. ID. NO: 81) |
| LGTAAESSWGDEQA | (SEQ. ID. NO: 82) |
| DEQADFRCDTIQP | (SEQ. ID. NO: 83) |
| TIQPGCQNVCTDQ | (SEQ. ID. NO: 84) |
| VCTDQAFPISHIR | (SEQ. ID. NO: 85) |
| AFPISHIRFWVLQ | (SEQ. ID. NO: 86) |
| MEVGFIVGQYFIYGIFLTTLHVCRRSPCPHPVNCYVSRPTEKNVFIV | (SEQ. ID. NO: 87) |
| MEVGFIVGQYF | (SEQ. ID. NO: 88) |
| IVGQYFIYGIFL | (SEQ. ID. NO: 89) |
| GIFLTTLHVCRRSP | (SEQ. ID. NO: 90) |
| RRSPCPHPVNCY | (SEQ. ID. NO: 91) |
| VNCYVSRPTEKN | (SEQ. ID. NO: 92) |
| SRPTEKNVFIV | (SEQ. ID. NO: 93) |

Table 7 provides peptides inhibitors of connexin 45 shown with reference to the extracellular loops (E1 and E2) of connexin 45. The bold amino acids are directed to the transmembrane regions of connexin 45

TABLE 7
Cx45 peptide inhibitors
E1

| | |
|---|---|
| LTAVGGESIYYDEQSKFVCNTEQPGCENVCYDAFAPLSHVRFWVFQ | (SEQ. ID. NO: 94) |
| LTAVGGESIYYDEQS | (SEQ. ID. NO: 95) |
| DEQSKFVCNTEQP | (SEQ. ID. NO: 96) |
| TEQPGCENVCYDA | (SEQ. ID. NO: 97) |
| VCYDAFAPLSHVR | (SEQ. ID. NO: 98) |
| APLSHVRFWVFQ | (SEQ. ID. NO: 99) |

E2

| | |
|---|---|
| FEVGFLIGQYFLYGFQVHPFYVCSRLPCHPKIDCFISRPTEKTIFLL | (SEQ. ID. NO: 100) |
| FEVGFLIGQYF | (SEQ. ID. NO: 101) |
| LIGQYFLYGFQV | (SEQ. ID. NO: 102) |
| GFQVHPFYVCSRLP | (SEQ. ID. NO: 103) |
| SRLPCHPKIDCF | (SEQ. ID. NO: 104) |
| IDCFISRPTEKT | (SEQ. ID. NO: 105) |
| SRPTEKTIFLL | (SEQ. ID. NO: 106) |

In certain embodiments, it is preferred that certain peptide inhibitors block hemichannels without disrupting existing gap junctions. While not wishing to be bound to any particular theory or mechanism, it is also believed that certain peptidomimetics (e.g. VCYDKSFPISHVR, (SEQ.ID.NO:23) block hemichannels without causing uncoupling of gap junctions (See Leybeart et al., Cell Commun. Adhes. 10: 251-257 (2003)), or do so in lower dose amounts. The peptide SRPTEKTIFII (SEQ.ID.NO:19) may also be used, for example to block hemichannels without uncoupling of gap junctions. The peptide SRGGEKNVFIV (SEQ.ID.NO:107) may be used that as a control sequence (DeVriese et al., Kidney Internat. 61: 177-185 (2002)). Examples of peptide inhibitors for connexin 45 YVCSRLPCHP (SEQ.ID.NO: 108), QVHPFYVCSRL (SEQ.ID.NO:109), FEVGFLIGQYFLY (SEQ.ID.NO: 110), GQYFLYGFQVHP (SEQ.ID.NO: 111), GFQVHPFYVCSR (SEQ.ID.NO:112), AVGGESIYYDEQ (SEQ.ID.NO.:113), YDEQSKFVCNTE (SEQ.ID.NO:114), NTEQPGCENVCY (SEQ.ID.NO:115), CYDAFAPLSHVR (SEQ.ID.NO:116), FAPLSHVRFWVF (SEQ.ID.NO:117) and LIGQY (SEQ.ID.NO:118), QVHPF (SEQ.ID.NO:119), YVCSR (SEQ.ID.NO:120), SRLPC (SEQ.ID.NO:121), LPCHP (SEQ.ID.NO:122) and GESIY (SEQ.ID.NO:123), YDEQSK (SEQ.ID.NO:124), SKFVCN (SEQ.ID.NO:125), TEQPGCEN (SEQ.ID.NO:126), VCYDAFAP (SEQ.ID.NO:127), LSHVRFWVFQ (SEQ.ID.NO:128) The peptides may only be 3 amino acids in length, including SRL, PCH, LCP, CHP, IYY, SKF, QPC, VCY, APL, HVR, or longer, for example: LIQYFLYGFQVHPF (SEQ.ID.NO:129), VHPFYCSRLPCHP (SEQ.ID.NO: 130), VGGESIYYDEQSKFVCNTEQPG (SEQ.ID.NO:131), TEQPGCENVCYDAFAPLSHVRF (SEQ.ID.NO: 132), AFAPLSHVRFWVFQ (SEQ.ID.NO: 133).

TABLE 8
Table 8A
Human Connexin 43 from GenBank Accession No. M65188 (SEQ. ID. NO: 134)

| | |
|---|---|
| 1 | ggcttttagc gtgaggaaag taccaaacag cagcggagtt ttaaacttta aatagacagg |
| 61 | tctgagtgcc tgaacttgcc ttttcatttt acttcatcct ccaaggagtt caatcacttg |
| 121 | gcgtgacttc actactttta agcaaaagag tggtgcccag gcaacatggg tgactggagc |
| 181 | gccttaggca aactccttga caaggttcaa gcctactcaa ctgctggagg gaaggtgtgg |
| 241 | ctgtcagtac ttttcatttt ccgaatcctg ctgctgggga cagcggttga gtcagcctgg |
| 301 | ggagatgagc agtctgcctt tcgttgtaac actcagcaac ctggttgtga aaatgtctgc |
| 361 | tatgacaagt ctttcccaat ctctcatgtg cgcttctggg tcctgcagat catatttgtg |
| 421 | tctgtaccca cactcttgta cctggctcat gtgttctatg tgatgcgaaa ggaagagaaa |
| 481 | ctgaacaaga aagaggaaga actcaaggtt gcccaaactg atggtgtcaa tgtggacatg |
| 541 | cacttgaagc agattgagat aaagaagttc aagtacggta ttgaagagca tggtaaggtg |
| 601 | aaaatgcgag gggggttgct gcgaacctac atcatcagta tcctcttcaa gtctatcttt |
| 661 | gaggtggcct tcttgctgat ccagtggtac atctatggat tcagcttgag tgctgtttac |
| 721 | acttgcaaaa gagatccctg cccacatcag gtggactgtt tcctctctcg ccccacggag |
| 781 | aaaaccatct tcatcatctt catgctggtg gtgtccttgg tgtccctggc cttgaatatc |
| 841 | attgaactct tctatgtttt cttcaagggc gttaaggatc gggttaaggg aaagagcgac |
| 901 | ccttaccatg cgaccagtgg tgcgctgagc cctgccaaag actgtgggtc tcaaaaatat |
| 961 | gcttatttca atggctgctc ctcaccaacc gctcccctct cgcctatgtc tcctcctggg |
| 1021 | tacaagctgg ttactggcga cagaaacaat tcttcttgcc gcaattacaa caagcaagca |
| 1081 | agtgagcaaa actgggctaa ttacagtgca gaacaaaatc gaatggggca ggcgggaagc |
| 1141 | accatctcta actcccatgc acagcctttt gatttccccg atgataacca gaattctaaa |
| 1201 | aaactagctg ctggacatga attacagcca ctagccattg tggaccagcg accttcaagc |
| 1261 | agagccagca gtcgtgccag cagcagacct cggcctgatg acctggagat ctag |

Table 8B
Human Connexin 43 (SEQ. ID. NO: 135)

| | |
|---|---|
| 1 | atgggtgactggagcgcctt aggcaaactc cttgacaagg ttcaagccta ctcaactgct |
| 61 | ggagggaaggtgtggctgtc agtacttttc attttccgaatcctgctgct ggggacagcg |
| 121 | gttgagtcagcctggggaga tgagcagtct gcctttcgtt gtaacactca gcaacctggt |
| 181 | tgtgaaaatg tctgctatga caagtctttcccaatctctc atgtgcgctt ctgggtcctg |
| 241 | cagatcatat ttgtgtctgt acccacactcttgtacctgg ctcatgtgttctatgtgatg |
| 301 | cgaaaggaag agaaactgaa caagaaagag gaagaactca aggttgccca aactgatggt |
| 361 | gtcaatgtgg acatgcactt gaagcagatt gagataaagaagttcaagta cggtattgaa |
| 421 | gagcatggta aggtgaaaat gcgagggggg ttgctgcgaa cctacatcat cagtatcctc |
| 481 | ttcaagtcta tctttgaggt ggccttcttg ctgatccagt ggtacatcta tggattcagc |
| 541 | ttgagtgctg tttacacttg caaaagagat ccctgcccac atcaggtgga ctgtttcctc |
| 601 | tctcgcccca cggagaaaac catcttcatc atcttcatgc tggtggtgtc cttggtgtcc |
| 661 | ctggccttga atatcattga actcttctat gttttcttca agggcgttaa ggatcgggtt |
| 721 | aagggaaaga gcgaccctta ccatgcgacc agtggtgcgc tgagccctgc caaagactgt |
| 781 | gggtctcaaa aatatgctta tttcaatggc tgctcctcac caaccgctcc cctctcgcct |
| 841 | atgtctcctc ctgggtacaa gctggttact ggcgacagaa acaattcttc ttgccgcaat |
| 901 | tacaacaagc aagcaagtga gcaaaactgg gctaattaca gtgcagaaca aaatcgaatg |
| 961 | gggcaggcgg gaagcaccat ctctaactcc catgcacagccttttgattt ccccgatgat |
| 1021 | aaccagaatt ctaaaaaactagctgctgga catgaattac agccactagc cattgtggac |
| 1081 | cagcgacctt caagcagagc cagcagtcgtgccagcagca gacctcggcctgatgacctg |
| 1141 | gagatctag |

### Gap Junction Modulation Agents

Certain anti-connexin agents described herein are capable of modulation or affecting the transport of molecules into and out of cells (e.g. blocking or inhibiting). Thus certain gap junction modulation agents described herein modulate cellular communication (e.g. cell to cell). Certain gap junction modulation agents modulate or affect transmission of molecules between the cell cytoplasm and the periplasmic or extracellular space. Such agents are generally targeted to hemichannels (also called connexins), which may be independently involved in the exchange of small molecules between the cell cytoplasm and an extracellular space or tissue. Thus, a compound provided herein may directly or indirectly reduce coupling between cells (via gap junctions) or between a cell and an extracellular space or tissue (via hemichannels), and the modulation of transport of molecules from a cell into an extracellular space is within the scope of certain compounds and embodiments of the invention.

Any molecule that is capable of eliciting a desired inhibition of the passage (e.g. transport) of molecules through a gap junction or hemichannel may be used in embodiments of the invention. Compounds that modulate the passage of molecules through a gap junction or hemichannel are also provided in particular embodiments (e.g., those that modulate the passage of molecules from the cytoplasm of a cell into an extracellular space). Such compounds may modulate the passage of molecules through a gap junction or hemichannel with or without gap junction uncoupling. Such compounds include, for example, binding proteins, polypeptides, and other organic compound that can, for example, block the function or activity of a gap junction or a hemichannel in whole or in part.

As used herein, “gap junction modulation agent” may broadly include those agents or compounds that prevent, decrease or modulate, in whole or in part, the activity, function, or formation of a hemichannel or a gap junction. In certain embodiments, a gap junction modulation agent prevents or decreases, in whole or in part, the function of a hemichannel or a gap junction. In certain embodiments, a gap junction modulation agent induces closure, in whole or in part, of a hemichannel or a gap junction. In other embodiments, a gap junction modulation agent blocks, in whole or in part, a hemichannel or a gap junction. In certain embodiments, a gap junction modulation agent decreases or prevents, in whole or in part, the opening of a hemichannel or gap junction. In certain embodiments, said blocking or closure of a gap junction or hemichannel by a gap junction modulation agent can reduce or inhibit extracellular hemichannel communication by preventing or decreasing the flow of small molecules through an open channel to and from an extracellular or periplasmic space. Peptidomimetics, and gap junction phosphorylation compounds that block hemichannel and/or gap junction opening are presently preferred.

In certain embodiments, a gap junction modulation agent prevents, decreases or alters the activity or function of a hemichannel or a gap junction. As used herein, modification of the gap junction activity or function may include the closing of gap junctions, closing of hemichannels, and/or passage of molecules or ions through gap junctions and/or hemichannels.

Exemplary gap junction modulation agents may include, without limitation, polypeptides (e.g. peptiditomimetics, antibodies, binding fragments thereof, and synthetic constructs), and other gap junction blocking agents, and gap junction protein phosphorylating agents. Exemplary compounds used for closing gap junctions (e.g. phosphorylating connexin 43 tyrosine residue) have been reported in U.S. Pat. No. 7,153,822 to Jensen et al., U.S. Pat. No. 7,250,397, and assorted patent publications. Exemplary peptides and peptidomimetics are reported in Green et al., WO2006134494. See also Gourdie et al., see WO2006069181, and Tudor et al., see WO2003032964.

As used herein, “gap junction phosphorylating agent” may include those agents or compounds capable of inducing phosphorylation on connexin amino acid residues in order to induce gap junction or hemichannel closure. Gap junction modulation exemplary sites of phosphorylation include one or more of a tyrosine, serine or threonine residues on the connexin protein. In certain embodiments, modulation of phosphorylation may occur on one or more residues on one or more connexin proteins. Exemplary gap junction phosphorylating agents are well known in the art and may include, for example, c-Src tyrosine kinase or other G protein-coupled receptor agonists. See Giepmans B (2001) J. Biol. Chem., Vol. 276, Issue 11, 8544-8549. In one embodiment, modulation of phosphorylation on one or more of these residues impacts hemichannel function, particularly by closing the hemichannel. In another embodiment, modulation of phosphorylation on one or more of these residues impacts gap junction function, particularly by closing the gap junction. Gap junction phosphorylating agents that target the closure of connexin 43 gap junctions and hemichannels are preferred.

Polypeptide compounds, including binding proteins (e.g. antibodies, antibody fragments, and the like), peptides, peptidomimetics, and peptidomimetics, are suitable modulators of gap junctions.

Binding proteins include, for example, monoclonal antibodies, polyclonal antibodies, antibody fragments (including, for example, Fab, F(ab′)2 and Fv fragments; single chain antibodies; single chain Fvs; and single chain binding molecules such as those comprising, for example, a binding domain, hinge, CH2 and CH3 domains, recombinant antibodies and antibody fragments which are capable of binding an antigenic determinant (i.e., that portion of a molecule, generally referred to as an epitope) that makes contact with a particular antibody or other binding molecule. These binding proteins, including antibodies, antibody fragments, and so on, may be chimeric or humanized or otherwise made to be less immunogenic in the subject to whom they are to be administered, and may be synthesized, produced recombinantly, or produced in expression libraries. Any binding protein known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g. connexin, connexon, gap junctions, or associated molecules).

Binding proteins will generally have a desired specificity, including but not limited to binding specificity, and desired affinity. Affinity, for example, may be a Ka of greater than or equal to about 104 M−1, greater than or equal to about 106 M−1, greater than or equal to about 107 M−1, greater than or equal to about 108 M−1. Affinities of even greater than about 108 M−1 are suitable, such as affinities equal to or greater than about 109 M−1, about 1010 M−1, about 1011 M−1, and about 1012 M−1. Affinities of binding proteins according to the present invention can be readily determined using conventional techniques, for example those described by Scatchard et al., (1949) Ann. N.Y. Acad. Sci. 51: 660.

The invention includes use of peptides (including peptidomimetic and peptidomimetics) for modulation of gap junctions and hemichannels. By using data obtained from hydropathy plots, it has been proposed that a connexin contains four-transmembrane-spanning regions and two short extra-cellular loops. The positioning of the first and second extracellular regions of connexin was further characterized by the reported production of anti-peptide antibodies used for immunolocalization of the corresponding epitopes on split gap junctions. Goodenough D. A. (1988) J Cell Biol 107: 1817-1824; Meyer R. A. (1992) J Cell Biol 119: 179-189.

Peptides or variants thereof, can be synthesized in vitro, e.g., by the solid phase peptide synthetic method or by enzyme-catalyzed peptide synthesis or with the aid of recombinant DNA technology. Solid phase peptide synthetic method is an established and widely used method, which is described in references such as the following: Stewart et al., (1969) Solid Phase Peptide Synthesis, W. H. Freeman Co., San Francisco; Merrifield, (1963) J. Am. Chem. Soc. 85 2149; Meienhofer in “Hormonal Proteins and Peptides,” ed.; C. H. Li, Vol. 2 (Academic Press, 1973), pp. 48-267; and Bavaay and Merrifield, “The Peptides,” eds. E. Gross and F. Meienhofer, Vol. 2 (Academic Press, 1980) pp. 3-285. These peptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; ligand affinity chromatography; or crystallization or precipitation from non-polar solvent or nonpolar/polar solvent mixtures. Purification by crystallization or precipitation is preferred.

The extracellular domains of a hemichannel contributed by two adjacent cells “dock” with each other to form complete gap junction channels. Reagents that interfere with the interactions of these extracellular domains can impair cell-to-cell communication, or with hemichannel opening to the extracellular environment.

Gap junction modulation agents include peptides comprising an amino acid sequence corresponding to a transmembrane region (e.g. 1st to 4th) of a connexin (e.g. connexin 45, 43, 26, 30, 31.1, and 37). Gap junction modulation agents including a peptide comprising an amino acid sequence corresponding to a portion of a transmembrane region of a connexin 43 are preferred for use in the present inventions.

Gap junction modulation agents may comprise a peptide comprising an amino acid sequence corresponding to a portion of a transmembrane region of a connexin 45. Gap junction modulation agents include a peptide having an amino acid sequence that comprises about 5 to 20 contiguous amino acids of SEQ.ID.NO:13, a peptide having an amino acid sequence that comprises about 8 to 15 contiguous amino acids of SEQ.ID.NO:13, or a peptide having an amino acid sequence that comprises about 11 to 13 contiguous amino acids of SEQ.ID.NO:13. Other embodiments are directed to an gap junction modulation compound that is a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ.ID.NO:13. In certain gap junction modulation compounds provided herein, the extracellular domains of connexin 45 corresponding to the amino acids at positions 46-75 and 199-228 of SEQ.ID.NO:13 may be used to develop the particular peptide sequences. Certain peptides described herein have an amino acid sequence corresponding to the regions at positions 46-75 and 199-228 of SEQ.ID.NO:13. The peptides need not have an amino acid sequence identical to those portions of SEQ.ID.NO:13, and conservative amino acid changes may be made such that the peptides retain binding activity or functional activity. Alternatively, the peptide may target regions of the connexin protein other than the extracellular domains (e.g. the portions of SEQ.ID.NO:13 not corresponding to positions 46-75 and 199-228).

Also, suitable gap junction modulation agents can include a peptide comprising an amino acid sequence corresponding to a portion of a transmembrane region of a connexin 43. Gap junction modulation agents include peptides having an amino acid sequence that comprises about 5 to 20 contiguous amino acids of SEQ.ID.NO:14, peptides having an amino acid sequence that comprises about 8 to 15 contiguous amino acids of SEQ.ID.NO:14, or peptides having an amino acid sequence that comprises about 11 to 13 contiguous amino acids of SEQ.ID.NO:14. Other gap junction modulation agents include a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of SEQ.ID.NO:14. Other gap junction modulation agents comprise the extracellular domains of connexin 43 corresponding to the amino acids at positions 37-76 and 178-208 of SEQ.ID.NO:14. Gap junction modulation agents include peptides described herein which have an amino acid sequence corresponding to the regions at positions 37-76 and 178-208 of SEQ.ID.NO:14. The peptides need not have an amino acid sequence identical to those portions of SEQ.ID.NO:14, and conservative amino acid changes may be made such that the peptides retain binding activity or functional activity. Alternatively, peptides may target regions of the connexin protein other than the extracellular domains (e.g. the portions of SEQ.ID.NO:14 not corresponding to positions 37-76 and 178-208).

Still other anti-connexin agents include connexin carboxy-terminal polypeptides. See Gourdie et al., WO2006/069181.

Gap Junction Modifying Agents—Other Anti-Connexin Agents

Gap junction modulation agents, include agents that close or block gap junctions and/or hemichannels or otherwise prevent or decrease cell to cell communication via gap junctions or prevent or decrease cell communication to the extracellular environment via hemichannels. They include agents or compounds that prevent, decrease or inhibit, in whole or in part, the activity, function, or formation of a hemichannel or a gap junction.

In certain embodiments, a gap junction modulation agent induces closure, in whole or in part, of a hemichannel or a gap junction. In other embodiments, a gap junction modifying agent blocks, in whole or in part, a hemichannel or a gap junction. In certain embodiments, a gap junction modifying agent decreases or prevents, in whole or in part, the opening of a hemichannel or gap junction.

In certain embodiments, said blocking or closure of a gap junction or hemichannel by a gap junction modifying agent can reduce or inhibit extracellular hemichannel communication by preventing or decreasing the flow of small molecules through an open channel to and from an extracellular or periplasmic space.

Gap junction modifying agents used for closing hemichannels or gap junctions (e.g. phosphorylating connexin 43 tyrosine residues) have been reported in U.S. Pat. No. 7,153,822 to Jensen et al., U.S. Pat. No. 7,250,397, and assorted patent publications. See also Gourdie et al., see WO2006069181, with regard to connexin carboxy-terminal polypeptides that are said to, for example, inhibit ZO-1 protein binding. Gourdie et al, WO2006069181 describes use of formulations comprising such peptides.

As used herein, “gap junction phosphorylating agent” may include those agents or compounds capable of inducing phosphorylation on connexin amino acid residues in order to induce gap junction or hemichannel closure. Exemplary sites of phosphorylation include one or more of a tyrosine, serine or threonine residues on the connexin protein. In certain embodiments, modulation of phosphorylation may occur on one or more residues on one or more connexin proteins. Exemplary gap junction phosphorylating agents are well known in the art and may include, for example, c-Src tyrosine kinase or other G protein-coupled receptor agonists. See Giepmans B, J. Biol. Chem., Vol. 276, Issue 11, 8544-8549, Mar. 16, 2001. In one embodiment, modulation of phosphorylation on one or more of these residues impacts hemichannel function, particularly by closing the hemichannel. In another embodiment, modulation of phosphorylation on one or more of these residues impacts gap junction function, particularly by closing the gap junction. Gap junction phosphorylating agents that target the closure of connexin 43 gap junctions and hemichannels are preferred.

Still other anti-connexin agents include connexin carboxy-terminal polypeptides. See Gourdie et al., WO2006/069181.

In certain another aspect, gap junction modifying agent may include, for example, aliphatic alcohols; octanol; heptanol; anesthetics (e.g. halothane), ethrane, fluothane, propofol and thiopental; anandamide; arylaminobenzoate (FFA: flufenamic acid and similar derivatives that are lipophilic); carbenoxolone; Chalcone: (2′,5′-dihydroxychalcone); CHFs (Chlorohydroxyfuranones); CMCF (3-chloro-4-(chloromethyl)-5-hydroxy-2(5H)-furanone); dexamethasone; doxorubicin (and other anthraquinone derivatives); eicosanoid thromboxane A(2) (TXA(2)) mimetics; NO (nitric oxide); Fatty acids (e.g. arachidonic acid, oleic acid and lipoxygenase metabolites; Fenamates (flufenamic (FFA), niflumic (NFA) and meclofenamic acids (MFA)); Genistein; glycyrrhetinic acid (GA): 18a-glycyrrhetinic acid and 18-beta-glycyrrhetinic acid, and derivatives thereof; lindane; lysophosphatidic acid; mefloquine; menadione; 2-Methyl-1,4-naphthoquinone, vitamin K(3); nafenopin; okadaic acid; oleamide; oleic acid; PH, gating by intracellular acidification; e.g. acidifying agents; polyunsaturated fatty acids; fatty acid GJIC inhibitors (e.g. oleic and arachidonic acids); quinidine; quinine; all trans-retinoic acid; and tamoxifen.

### Dosage Forms and Formulations and Administration

A therapeutically effective amount of each of the combination partners (e.g. an anti-connexin polynucleotide and an anti-connexin peptide or peptidomimetic) may be administered simultaneously, separately or sequentially and in any order. The agents may be administered separately or as a fixed combination. When not administered as a fixed combination, preferred methods include the sequential administration of one or more anti-connexin polynucleotides and one or more anti-connexin peptides or peptidomimetics, either or both of which are provided in amounts or doses that are less that those used when the agent or agents are administered alone, i.e., when they are not administered in combination, either physically or in the course of treatment of a wound. Such lesser amounts of agents administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone. Preferably, the agents are administered sequentially within at least about one-half hour of each other. The agents may also be administered with about one hour of each other, with about one day to about one week of each other, or as otherwise deemed appropriate. Preferably, an anti-connexin peptide or anti-connexin peptidomimetic, e.g., an anti-connexin agent that can block or reduce hemichannel opening, is administered prior to the administration of an anti-connexin agent that blocks or reduce connexin expression or the formation of hemichannels or gap junctions, e.g., by downregulation of connexin protein expression. Preferably, the anti-connexin agent or agents is/are anti-connexin 43 agent(s).

The agents of the invention of the may be administered to a subject in need of treatment, such as a subject with any of the diseases or conditions mentioned herein. The condition of the subject can thus be improved. The anti-connexin agents may thus be used in the treatment of the subject's body by therapy. They may be used in the manufacture of a medicament to treat any of the conditions mentioned herein. Thus, in accordance with the invention, there are provided formulations by which cell-cell communication can be downregulated in a transient and site-specific manner.

The anti-connexin agent may be present in a substantially isolated form. It will be understood that the product may be mixed with carriers or diluents which will not interfere with the intended purpose of the product and still be regarded as substantially isolated. A product of the invention may also be in a substantially purified form, in which case it will generally comprise about 80%, 85%, or 90%, e.g. at least about 95%, at least about 98% or at least about 99% of the polynucleotide (or other anti-connexin agent) or dry mass of the preparation.

Depending on the intended route of administration, the pharmaceutical products, pharmaceutical compositions, combined preparations and medicaments of the invention may, for example, take the form of solutions, suspensions, instillations, salves, creams, gels, foams, ointments, emulsions, lotions, paints, sustained release formulations, or powders, and typically contain about 0.1%-95% of active ingredient(s), preferably about 0.2%-70%. Other suitable formulations include pluronic gel-based formulations, carboxymethylcellulose (CMC)-based formulations, and hyroxypropylmethylcellulose (HPMC)-based formulations. Suitable formulations including pluronic gel, have for example about 10 to about 15 percent, suitably about 12 percent, pluronic gel. Other useful formulations include slow or delayed release preparations.

Gels or jellies may be produced using a suitable gelling agent including, but not limited to, gelatin, tragacanth, or a cellulose derivative and may include glycerol as a humectant, emollient, and preservative. Ointments are semi-solid preparations that consist of the active ingredient incorporated into a fatty, waxy, or synthetic base. Examples of suitable creams include, but are not limited to, water-in-oil and oil-in-water emulsions. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax. Suitable properties include the ability to modify the viscosity of the emulsion and both physical and chemical stability over a wide range of pH. The water soluble or miscible cream base may contain a preservative system and may also be buffered to maintain an acceptable physiological pH.

Foam preparations may be formulated to be delivered from a pressurized aerosol canister, via a suitable applicator, using inert propellants. Suitable excipients for the formulation of the foam base include, but are not limited to, propylene glycol, emulsifying wax, cetyl alcohol, and glyceryl stearate. Potential preservatives include methylparaben and propylparaben.

Preferably the agents of the invention are combined with a pharmaceutically acceptable carrier or diluent to produce a pharmaceutical composition. Suitable carriers and diluents include isotonic saline solutions, for example phosphate-buffered saline. Suitable diluents and excipients also include, for example, water, saline, dextrose, glycerol, or the like, and combinations thereof. In addition, if desired substances such as wetting or emulsifying agents, stabilizing or ph buffering agents may also be present.

The term “pharmaceutically acceptable carrier” refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, and amino acid copolymers.

Pharmaceutically acceptable salts can also be present, e.g., mineral acid salts such as hydrochlorides, hydrobromides, phosphates, sulfates, and the like; and the salts of organic acids such as acetates, propionates, malonates, benzoates, and the like.

Suitable carrier materials include any carrier or vehicle commonly used as a base for creams, lotions, gels, emulsions, lotions or paints for topical administration. Examples include emulsifying agents, inert carriers including hydrocarbon bases, emulsifying bases, non-toxic solvents or water-soluble bases. Particularly suitable examples include pluronics, HPMC, CMC and other cellulose-based ingredients, lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol and stearyl alcohol.

Preferably, the pharmaceutically acceptable carrier or vehicle is a gel, suitably a nonionic polyoxyethylene-polyoxypropylene copolymer gel, for example, a Pluronic gel, preferably Pluronic F-127 (BASF Corp.). This gel is particularly preferred as it is a liquid at low temperatures but rapidly sets at physiological temperatures, which confines the release of the agent to the site of application or immediately adjacent that site.

An auxiliary agent such as casein, gelatin, albumin, glue, sodium alginate, carboxymethylcellulose, methylcellulose, hydroxyethylcellulose or polyvinyl alcohol may also be included in the formulation of the invention.

Other suitable formulations include pluronic gel-based formulations, carboxymethylcellulose (CMC)-based formulations, and hyroxypropylmethylcellulose (HPMC)-based formulations. The composition may be formulated for any desired form of delivery, including topical, instillation, parenteral, intramuscular, subcutaneous, or transdermal administration. Other useful formulations include slow or delayed release preparations.

Where the anti-connexin agent is a nucleic acid, such as a polynucleotide, uptake of nucleic acids by mammalian cells is enhanced by several known transfection techniques for example those including the use of transfection agents. Such techniques may be used with certain anti-connexin agents, including polynucleotides. The formulation which is administered may contain such transfection agents. Examples of these agents include cationic agents (for example calcium phosphate and DEAE-dextran) and lipofectants (for example lipofectam™ and transfectam™), and surfactants.

Where the anti-connexin agent comprises a polynucleotide, conveniently, the formulation further includes a surfactant to assist with polynucleotide cell penetration or the formulation may contain any suitable loading agent. Any suitable non-toxic surfactant may be included, such as DMSO. Alternatively a transdermal penetration agent such as urea may be included.

The effective dose for a given subject or condition can be determined by routine experimentation or other methods known in the art or later developed. For example, in order to formulate a range of dosage values, cell culture assays and animal studies can be used. The dosage of such compounds preferably lies within the dose that is therapeutically effective for at least 50% of the population, and that exhibits little or no toxicity at this level.

The effective dosage of each of the anti-connexin agents employed in the methods and compositions of the invention may vary depending on a number of factors including the particular anti-connexin agent or agents employed, the combinational partner, the mode of administration, the frequency of administration, the condition being treated, the severity of the condition being treated, the route of administration, the needs of a patient sub-population to be treated or the needs of the individual patient which different needs can be due to age, sex, body weight, relevant medical condition specific to the patient.

The dose at which an anti-connexin agent is administered to a patient will depend upon a variety of factors such as the age, weight and general condition of the patient, the condition that is being treated, and the particular anti-connexin agent that is being administered.

A suitable therapeutically effective dose of an anti-connexin agent may be from about 0.001 to about 1 mg/kg body weight such as about 0.01 to about 0.4 mg/kg body weight. A suitable dose may however be from about 0.001 to about 0.1 mg/kg body weight such as about 0.01 to about 0.050 mg/kg body weight.

Therapeutically effective doses of anti-connexin agents from about 1 to 100, 100-200, 100- or 200-300, 100- or 200- or 300-400, and 100- or 200- or 300- or 400-500 micrograms are appropriate. Doses from about 1-1000 micrograms are also appropriate. Doses up to 2 milligrams may also be used. Doses are adjusted appropriately when the anti-connexin agent or agents are provided in the form of a dressing, typically upward to maintain the desired total dose administration.

Alternatively, in the case of anti-connexin oligonucleotides or anti-connexin peptidomimetics, the dosage of each of the gap junction modulation agents in the compositions may be determined by reference to the composition's concentration relative to the size, length, depth, area or volume of the area to which it will be applied. For example, in certain topical applications, dosing of the pharmaceutical compositions may be calculated based on mass (e.g. grams) of or the concentration in a pharmaceutical composition (e.g. μg/ul) per length, depth, area, or volume of the area of application. Useful doses range from about 1 to about 10 micrograms per square centimeter of wound size. Certain doses will be about 1-2, about 1-5, about 2-4, about 5-7, and about 8-10 micrograms per square centimeter of wound size. Other useful doses are greater than about 10 micrograms per square centimeter of wound size, including at least about 15 micrograms per square centimeter of wound size, at least about 20 micrograms per square centimeter of wound size, at least about 25 micrograms per square centimeter of wound size, about 30 micrograms per square centimeter of wound size, at least about 35 micrograms per square centimeter of wound size, at least about 40 micrograms per square centimeter of wound size, at least about 50 micrograms per square centimeter of wound size, and at least about 100 to at least about 150 micrograms per square centimeter of wound size. Other doses include about 150-200 micrograms per square centimeter, about 200-250 micrograms per square centimeter, about 250-300 micrograms per square centimeter, about 300-350 micrograms per square centimeter, about 350-400 micrograms per square centimeter, and about 400-500 micrograms per square centimeter.

In certain embodiments, the anti-connexin agent composition may be applied at about 0.01 micromolar (μM) or 0.05 μM to about 200 μM, or up to 300 μM or up to 1000 μM or up to 2000 μM or up to 3200 μM or more final concentration at the treatment site and/or adjacent to the treatment site, and any doses and dose ranges within these dose numbers. Preferably, the antisense polynucleotide composition is applied at about 0.05 μM to about 100 μM final concentration, more preferably, the anti-connexin agent composition is applied at about 1.0 μM to about 50 μM final concentration, and more preferably, the anti-connexin agent composition is applied at about 5-10 μM to about 30-50 μM final concentration. Additionally, the combined anti-connexin agent composition is applied at about 8 μM to about 20 μM final concentration, and alternatively the anti-connexin agent composition is applied at about 10 μM to about 20 μM final concentration, or at about 10 to about 15 μM final concentration. In certain other embodiments, the anti-connexin agent is applied at about 10 μM final concentration. In yet another embodiment, the anti-connexin agent composition is applied at about 1-15 μM final concentration. In other embodiments, the anti-connexin agent is applied at about a 20 μM, 30 μM, 40 μM, 50 μM, 60 μM, 70 μM, 80 μM, 90 μM, 100 μM, 10-200 μM, 200-300 μM, 300-400 μM, 400-500 μM, 500-600 μM, 600-700 μM, 700-800 μM, 800-900 μM, 900-1000 or 1000-1500 μM, or 1500 μM-2000 μM or 2000 μM-3000 μM or greater.

Anti-connexin agent dose amounts include, for example, about 0.1-1, 1-2, 2-3, 3-4, or 4-5 micrograms (μg), from about 5 to about 10 μg, from about 10 to about 15 μg, from about 15 to about 20 μg, from about 20 to about 30 μg, from about 30 to about 40 μg, from about 40 to about 50 μg, from about 50 to about 75 μg, from about 75 to about 100 μg, from about 100 μg to about 250 μg, and from 250 μg to about 500 μg. Dose amounts from 0.5 to about 1.0 milligrams or more or also provided, as noted above. Dose volumes will depend on the size of the site to be treated, and may range, for example, from about 25-100 μL to about 100-200 μL, from about 200-500 μL to about 500-1000 μL. Milliliter doses are also appropriate for larger treatment sites.

Still other dosage levels between about 1 nanogram (ng)/kg and about 1 mg/kg body weight per day of each of the agents described herein. In certain embodiments, the dosage of each of the subject compounds will generally be in the range of about 1 ng to about 1 microgram per kg body weight, about 1 ng to about 0.1 microgram per kg body weight, about 1 ng to about 10 ng per kg body weight, about 10 ng to about 0.1 microgram per kg body weight, about 0.1 microgram to about 1 microgram per kg body weight, about 20 ng to about 100 ng per kg body weight, about 0.001 mg to about 0.01 mg per kg body weight, about 0.01 mg to about 0.1 mg per kg body weight, or about 0.1 mg to about 1 mg per kg body weight. In certain embodiments, the dosage of each of the subject compounds will generally be in the range of about 0.001 mg to about 0.01 mg per kg body weight, about 0.01 mg to about 0.1 mg per kg body weight, about 0.1 mg to about 1 mg per kg body weight. If more than one anti-connexin agent is used, the dosage of each anti-connexin agent need not be in the same range as the other. For example, the dosage of one anti-connexin agent may be between about 0.01 mg to about 10 mg per kg body weight, and the dosage of another anti-connexin agent may be between about 0.1 mg to about 1 mg per kg body weight.

All doses and dose ranges referenced herein are applicable, for example, to anti-connexin oligonucleotides. These dose ranges are also applicable, for example, to anti-connexin peptides anti-connexin mimetic peptides and anti-connexin peptidomimetics.

Conveniently, the anti-connexin agent is administered in a sufficient amount to downregulate expression of a connexin protein, or modulate gap junction formation or connexon opening for at least about 0.5 to 1 hour, at least about 1-2 hours, at least about 2-4 hours, at least about 4-6 hours, at least about 6-8 hours, at least about 8-10 hours, at least about 12 hours, or at least about 24 hours post-administration.

The dosage of each of the anti-connexin agents in the compositions and methods of the subject invention may also be determined by reference to the concentration of the composition relative to the size, length, depth, area or volume of the area to which it will be applied. For example, in certain topical and other applications, e.g., instillation, dosing of the pharmaceutical compositions may be calculated based on mass (e.g. micrograms) of or the concentration in a pharmaceutical composition (e.g. μg/μl) per length, depth, area, or volume of the area of application.

As noted herein, the doses of an anti-connexin polynucleotide, peptide or peptidomimetic administered in combination, or other anti-connexin agents administered in combination with either or both, can be adjusted down from the doses administered when given alone.

The combined use of several agents may reduce the required dosage for any individual agent because the onset and duration of effect of the different agents may be complementary. In a preferred embodiment, the combined use of two or more anti-connexin agents has an additive, synergistic or super-additive effect.

In some cases, the combination of one or more anti-connexin polynucleotide and one or more anti-connexin peptides or peptidomimetics, or other anti-connexin agents administered in combination with either or both, have an additive effect. In other cases, the combination can have greater-than-additive effect. Such an effect is referred to herein as a “supra-additive” effect, and may be due to synergistic or potentiated interaction.

The term “supra-additive promotion of wound healing” refers to a mean wound healing produced by administration of a combination of one or more anti-connexin polynucleotides and one or more anti-connexin peptides or peptidomimetics, or other anti-connexin agents administered in combination with either or both, is statistically significantly higher than the sum of the wound healing produced by the individual administration of either of the agents alone. Whether produced by combination administration of one or more anti-connexin polynucleotides and one or more anti-connexin peptides or peptidomimetics, or other anti-connexin agents administered in combination with either or both, is “statistically significantly higher” than the expected additive value of the individual compounds may be determined by a variety of statistical methods as described herein and/or known by one of ordinary skill in the art. The term “synergistic” refers to a type of supra-additive inhibition in which both the anti-connexin polynucleotide and anti-connexin peptide or peptidomimetic, or other anti-connexin agents administered in combination with either or both, individually have the ability to promote wound healing. The term “potentiated” refers to type of supra-additive effect in which one of the anti-connexin polynucleotide, anti-connexin peptides or peptidomimetics, or other anti-connexin agents administered in combination with either or both, individually has the increased ability to promote wound healing.

In general, potentiation may be assessed by determining whether the combination treatment produces a mean wound healing increase in a treatment group that is statistically significantly supra-additive when compared to the sum of the mean wound healing increases produced by the individual treatments in their treatment groups respectively. The mean wound healing increase may be calculated as the difference between control group and treatment group mean wound healing. The fractional increase in wound healing, “fraction affected” (Fa), may be calculated by dividing the treatment group mean wound healing increase by control group mean wound healing. Testing for statistically significant potentiation requires the calculation of Fa for each treatment group. The expected additive Fa for a combination treatment may be taken to be the sum of mean Fas from groups receiving either element of the combination. The Two-Tailed One-Sample T-Test, for example, may be used to evaluate how likely it is that the result obtained by the experiment is due to chance alone, as measured by the p-value. A p-value of less than 0.05 is considered statistically significant, that is, not likely to be due to chance alone. Thus, Fa for the combination treatment group must be statistically significantly higher than the expected additive Fa for the single element treatment groups to deem the combination as resulting in a potentiated supra-additive effect.

Whether a synergistic effect results from a combination treatment may be evaluated by the median-effect/combination-index isobologram method (Chou, T., and Talalay, P. (1984) Ad. Enzyme Reg. 22:27-55). In this method, combination index (CI) values are calculated for different dose-effect levels based on parameters derived from median-effect plots of the anti-connexin agent alone, the one or more agents useful for wound healing alone, and the combination of the two at fixed molar ratios. CI values of & lt; 1 indicate synergy, CI-1 indicates an additive effect, and CP1 indicates an antagonistic effect. This analysis may be performed using computer software tools, such as CalcuSyn, Windows Software for Dose Effect Analysis (Biosoft(D, Cambridge UK).

Any method known or later developed in the art for analyzing whether a supra-additive effect exists for a combination therapy is contemplated for use in screening for suitable anti-connexin agents for use in combination.

In another preferred embodiment, the combined use of one or more anti-connexin polynucleotides and one or more anti-connexin peptides or peptidomimetics reduces the effective dose of any such agent compared to the effective dose when said agent administered alone. In certain embodiments, the effective dose of the agent when used in combination is about 1/15 to about ½, about 1/10 to about ⅓, about ⅛ to about ⅙, about ⅕, about ¼, about ⅓ or about ½ the dose of the agent when used alone.

In another preferred embodiment, the combined use of one or more anti-connexin polynucleotides and one or more anti-connexin peptides or peptidomimetics, or other anti-connexin agents in combination with either or both, reduces the frequency in which said agent is administered compared to the frequency when said agent is administered alone. Thus, these combinations allow the use of lower and/or fewer doses of each agent than previously required to achieve desired therapeutic goals.

The doses may be administered in single or divided applications. The doses may be administered once, or application may be repeated. Typically, application will be repeated weekly until wound healing is promoted, or a repeat application may be made in the event that wound healing slows or is stalled. Doses may be applied 3-7 days apart, or more. In the case of a chronic wound, repeat applications may be made, for example, weekly, or bi-weekly, or monthly or in other frequency for example if and when wound healing slows or is stalled. For some indications, such as certain ocular uses, more frequent dosing, up to hourly may employed.

One or more anti-connexin polynucleotides and one or more anti-connexin peptides or peptidomimetics may be administered by the same or different routes. The various agents of the invention can be administered separately at different times during the course of therapy, or concurrently in divided or single combination forms.

In one aspect of the invention the anti-connexin polynucleotide is administered in one composition and the anti-connexin peptide or peptidomimetic is administered in a second composition. In one embodiment the first composition comprising one or more anti-connexin peptide or peptidomimetics is administered before the second composition comprising one or more anti-connexin polynucleotides. In one embodiment the first composition comprising one or more anti-connexin peptides or peptidomimetics is administered after the second composition comprising one or more anti-connexin polynucleotides. In one embodiment the first composition comprising one or more anti-connexin peptides or peptidomimetics is administered before and after the second composition comprising one or more anti-connexin polynucleotides. In one embodiment the second composition comprising one or more anti-connexin polynucleotides is administered before and after the first composition comprising one or more anti-connexin peptides or peptidomimetics. In one embodiment the first composition comprising one or more anti-connexin peptides or peptidomimetics is administered about the same time as the second composition comprising one or more anti-connexin polynucleotides.

Preferably one or more anti-connexin polynucleotides and one or more anti-connexin peptides or peptidomimetics, or other anti-connexin agents administered in combination with either or both, are delivered by topical administration (peripherally or directly to a site), including but not limited to topical administration using solid supports (such as dressings and other matrices) and medicinal formulations (such as gels, mixtures, suspensions and ointments). In one embodiment, the solid support comprises a biocompatible membrane or insertion into a treatment site. In another embodiment, the solid support comprises a dressing or matrix. In one embodiment of the invention, the solid support composition may be a slow release solid support composition, in which the one or more anti-connexin polynucleotides and one or more anti-connexin peptides or peptidomimetics, or other anti-connexin agents to be administered in combination with either or both, is dispersed in a slow release solid matrix such as a matrix of alginate, collagen, or a synthetic bioabsorbable polymer. Preferably, the solid support composition is sterile or low bio-burden. In one embodiment, a wash solution comprising two or more anti-connexin agents can be used.

The delivery of a formulation comprising one or more anti-connexin polynucleotides and one or more anti-connexin peptides or peptidomimetics, or other anti-connexin agents to be administered in combination with either or both, over a period of time, in some instances for about 1-2 hours, about 2-4 hours, about 4-6 hours, about 6-8, or about 24 hours or longer, may be a particular advantage in more severe injuries or conditions. In some instances, cell loss may extend well beyond the site of a procedure to surrounding cells. Such loss may occur within 24 hours of the original procedure and is mediated by gap junction cell-cell communication, or hemichannel opening. Administration of anti-connexin agent(s), e.g., for downregulation of connexin expression, or blockade or inhibition of connexon opening or activity, therefore will modulate communication between the cells, or loss into the extracellular space in the case of connexon regulation, and minimize additional cell loss or injury or consequences of injury.

While the delivery period will be dependent upon both the site at which the downregulation is to be induced and the therapeutic effect which is desired, continuous or slow-release delivery for about 0.5-1 hour, about 1-2 hours, about 2-4 hours, about 4-6 hours, about 6-8, or about 24 hours or longer is provided. In accordance with the present invention, this is achieved by inclusion of one or more anti-connexin polynucleotides and one or more anti-connexin peptides or peptidomimetics, or other anti-connexin agents in combination with either or both, in a formulation together with a pharmaceutically acceptable carrier or vehicle, particularly in the form of a formulation for continuous or slow-release administration.

As noted, the one or more agents of the invention may be administered before, during, immediately following wounding, for example, or within about 180, about 120, about 90, about 60, or about 30 days, but preferably within about 10, about 9, about 8, about 7, about 6, about 5, about 4, about 3, or about 2 days or less, and most preferably within about 24, about 12, about 10, about 9, about 8, about 7, about 6, about 5, about 4, about 3, about 2, hours or within about 60, about 45, about 30, about 15, about 10, about 5, about 4, about 3, about 2, about 1 minute following wounding, for example.

The routes of administration and dosages described herein are intended only as a guide since a skilled physician will determine the optimum route of administration and dosage for any particular patient and condition.

Any of the methods of treating a subject having a wound and/or condition referenced or described herein may utilize the administration of any of the doses, dosage forms, formulations, and/or compositions herein described.

Dressings and Matrices

In one aspect, one or more anti-connexin polynucleotides and/or one or more anti-connexin peptides or peptidomimetics are provided in the form of a dressing or matrix. In certain embodiments, the one or more agents of the invention are provided in the form of a liquid, semi solid or solid composition for application directly, or the composition is applied to the surface of, or incorporated into, a solid contacting layer such as a dressing gauze or matrix. The dressing composition may be provided for example, in the faun of a fluid or a gel. One or more anti-connexin polynucleotides and one or more anti-connexin peptides or peptidomimetics may be provided in combination with conventional pharmaceutical excipients for topical application. Suitable carriers include: Pluronic gels, Polaxamer gels, Hydrogels containing cellulose derivatives, including hydroxyethyl cellulose, hydroxymethyl cellulose, carboxymethyl cellulose, hydroxypropylmethyl cellulose and mixtures thereof; and hydrogels containing polyacrylic acid (Carbopols). Suitable carriers also include creams/ointments used for topical pharmaceutical preparations, e.g., creams based on cetomacrogol emulsifying ointment. The above carriers may include alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilizers such as EDTA.

In addition to the biological matrices previously mentioned, suitable dressings or matrices may include, for example, the following with one or more anti-connexin polynucleotides and one or more anti-connexin peptides or peptidomimetics (or other anti-connexin agents to be administered in combination with either or both):

1) Absorptives: suitable absorptives may include, for example, absorptive dressings, which can provide, for example, a semi-adherent quality or a non-adherent layer, combined with highly absorptive layers of fibers, such as for example, cellulose, cotton or rayon. Alternatively, absorptives may be used as a primary or secondary dressing.

2) Alginates: suitable alginates include, for example, dressings that are non-woven, non-adhesive pads and ribbons composed of natural polysaccharide fibers or xerogel derived from seaweed. Suitable alginates dressings may, for example, form a moist gel through a process of ion exchange upon contact with exudate. In certain embodiments, alginate dressings are designed to be soft and conformable, easy to pack, tuck or apply over irregular-shaped areas. In certain embodiments, alginate dressings may be used with a second dressing.

3) Antimicrobial Dressings: suitable antimicrobial dressings may include, for example, dressings that can facilitate delivery of bioactive agents, such as, for example, silver and polyhexamethylene biguanide (PHMB), to maintain efficacy against infection, where this is needed or desirable. In certain embodiments, suitable antimicrobial dressings may be available as for example, as sponges, impregnated woven gauzes, film dressings, absorptive products, island dressings, nylon fabric, non-adherent barriers, or a combination of materials.

4) Biological & Biosynthetics: suitable biological dressings or biosynthetic dressings may include, for example, gels, solutions or semi-permeable sheets derived from a natural source, e.g., pigs or cows. In certain embodiments, a gel or solution is applied to the treatment site and covered with a dressing for barrier protection. In another embodiment, a biological-based (e.g., pig intestinal mucosa or bladder tissue) or biosynthetic-based sheet is placed in situ which may act as membrane, remaining in place after a single application, or the may be biological dressings or biosynthetic dressings may be prepared in advance to include one or more, preferably two, anti-connexin agents.

5) Collagens: suitable collagen dressings may include, for example, gels, pads, particles, pastes, powders, sheets or solutions derived from for example, bovine, porcine or avian sources or other natural sources or donors. In certain embodiments, the collagen dressing may interact with treatment site exudate to form a gel. In certain embodiments, collagen dressing may be used in combination with a secondary dressing.

6) Composites: suitable composite dressings may include, for example, dressings that combine physically distinct components into a single product to provide multiple functions, such as, for example, a bacterial barrier, absorption and adhesion. In certain embodiment, the composite dressings are comprised of, for example, multiple layers and incorporate a semi-or non-adherent pad. In certain embodiment, the composite may also include for example, an adhesive border of non-woven fabric tape or transparent film. In certain other embodiment, the composite dressing may function as for example, either a primary or a secondary dressing and in yet another embodiment, the dressing may be used in combination with topical pharmaceutical composition.

7) Contact Layers: suitable contact layer dressings may include, for example, thin, non-adherent sheets placed on an area to protect tissue from for example, direct contact with other agents or dressings applied to the treatment site. In certain embodiments, contact layers may be deployed to conform to the shape of the area of the treatment site and are porous to allow exudate to pass through for absorption by an overlying, secondary dressing. In yet another embodiment, the contact layer dressing may be used in combination with topical pharmaceutical composition.

8) Elastic Bandages: suitable elastic bandages may include, for example, dressings that stretch and conform to the body contours. In certain embodiment, the fabric composition may include for example, cotton, polyester, rayon or nylon. In certain other embodiments, the elastic bandage may for example, provide absorption as a second layer or dressing, to hold a cover in place, to apply pressure or to cushion a treatment site.

9) Foams: suitable foam dressings may include, for example, sheets and other shapes of foamed polymer solutions (including polyurethane) with small, open cells capable of holding fluids. Exemplary foams may be for example, impregnated or layered in combination with other materials. In certain embodiment, the absorption capability may be adjusted based on the thickness and composition of the foam. In certain other embodiments, the area in contact with the treatment site may be non-adhesive for easy removal. In yet another embodiment, the foam may be used in combination with an adhesive border and/or a transparent film coating that can serve as an anti-infective barrier.

10) Gauzes & Non-Woven dressings: suitable gauze dressings and woven dressings may include, for example, dry woven or non-woven sponges and wraps with varying degrees of absorbency. Exemplary fabric composition may include, for example, cotton, polyester or rayon. In certain embodiment, gauzes and non-woven dressing may be available sterile or non-sterile in bulk and with or without an adhesive border. Exemplary gauze dressings and woven dressings may be used for cleansing, packing and covering a variety of treatment sites.

11) Hydrocolloids: suitable hydrocolloid dressings may include, for example, wafers, powders or pastes composed of gelatin, pectin or carboxymethylcellulose. In certain embodiment, wafers are self-adhering and available with or without an adhesive border and in a wide variety of shapes and sizes. Exemplary hydrocolloids are useful on areas that require contouring. In certain embodiments, powders and pastes hydrocolloids may use used in combination with a secondary dressing.

12) Hydrogels (Amorphous): suitable amorphous hydrogel dressings may include, for example, formulations of water, polymers and other ingredients with no shape, designed to donate moisture and to maintain a moist healing environments and or to rehydrate the treatment site. In certain embodiment, hydrogels may be used in combination with a secondary dressing cover.

13) Hydrogels: Impregnated Dressings: suitable impregnated hydrogel dressings may include, for example, gauzes and non-woven sponges, ropes and strips saturated with an amorphous hydrogel. Amorphous hydrogels may include for example, formulations of water, polymers and other ingredients with no shape, designed to donate moisture to a dry treatment site and to maintain a moist healing environment.

14) Hydrogel Sheets: suitable hydrogel sheets may include for example, three-dimensional networks of cross-linked hydrophilic polymers that are insoluble in water and interact with aqueous solutions by swelling. Exemplary hydrogels are highly conformable and permeable and can absorb varying amounts of drainage, depending on their composition. In certain embodiment, the hydrogel is non-adhesive against the treatment site or treated for easy removal.

15) Impregnated Dressings: suitable impregnated dressings may include, for example, gauzes and non-woven sponges, ropes and strips saturated with a solution, an emulsion, oil, gel or some other pharmaceutically active compound or carrier agent, including for example, saline, oil, zinc salts, petrolatum, xeroform and scarlet red as well as the compounds described herein.

16) Silicone Gel Sheets: suitable silicone gel sheet dressings may include, for example, soft covers composed of cross-linked polymers reinforced with or bonded to mesh or fabric.

17) Solutions: suitable liquid dressings may include, for example, mixtures of multiprotein material and other elements found in the extracellular matrix. In certain embodiment, exemplary solutions may be applied to the treatment site after debridement and cleansing and then covered with an absorbent dressing or a nonadherent pad.

18) Transparent Films: suitable transparent film dressings may include polymer membranes of varying thickness coated on one side with an adhesive. In certain embodiments, transparent films are impermeable to liquid, water and bacteria but permeable to moisture vapor and atmospheric gases. In certain embodiments, the transparency allows visualization of the treatment site.

19) Fillers: suitable filler dressings may include, for example, beads, creams, foams, gels, ointments, pads, pastes, pillows, powders, strands or other formulations. In certain embodiment, fillers are non-adherent and may include a time-released antimicrobial. Exemplary fillers may be useful to maintain a moist environment, manage exudate, and for treatment of for example, partial- and full-thickness wounds, infected wounds, draining wounds and deep wounds that require packing.

### Combination Wound Treatment

General Aspects

The present invention is directed to pharmaceutical compositions and their methods of use wherein the composition comprises therapeutically effective amounts of one or more anti-connexin polynucleotides and one or more anti-connexin peptides or peptidomimetics, or other anti-connexin agents in combination with one or more of an anti-connexin polynucleotide and/or an anti-connexin peptide or peptidomimetic. The compositions are useful in enhancing or promoting healing of wounds, including acute wounds and wounds that do not heal at expected rates, such as chronic wounds and other wounds that may be slow to heal or refractory to conventional wound treatment or wound healing promoting therapies.

Equally, in instances of other tissue damage (particularly wounds) the methods and compositions of the invention are effective in promoting the wound healing process, reducing swelling and inflammation, and in minimizing scar formation. The formulations have clear benefit in the treatment of wounds, whether the result of external trauma (including burns), internal trauma, or surgical intervention, as well as chronic wounds.

Compositions

Accordingly, in one aspect, the invention provides compositions for use in therapeutic treatment, which comprises: at least one anti-connexin polynucleotide and at least one anti-connexin peptide or peptidomimetic, or other anti-connexin agents to be administered in combination with either or both or alone. In a preferred embodiment, the composition further comprises a pharmaceutically acceptable carrier or vehicle.

In one preferred form, the composition contains one or more antisense polynucleotides to the mRNA of one connexin protein only. In another preferred form, the composition comprises one or more anti-connexin peptides or peptidomimetics, or a gap junction or hemichannel phosphorylation agent or connexin carboxy-terminal polypeptide. Most preferably, this connexin protein is connexin 43.

In another preferred form, the composition comprises an anti-connexin peptide or peptidomimetic and an antisense polynucleotide to the mRNA of a connexin protein. Most preferably, this connexin is connexin 43.

### Treatment

General Aspects

The present invention is directed to pharmaceutical compositions and their methods of use for treating a subject during or following an orthopedic procedure or surgery or suffering from, predisposed to, or at risk for various orthopedic-related diseases, disorders, or conditions comprising therapeutically effective amounts of one or more peptides or peptidomimetics, or one or more anti-connexin polynucleotides and one or more anti-connexin peptides, peptidomimetics, or gap junction modifying agents, or other anti-connexin agents in combination with one or more of an anti-connexin polynucleotide and/or an anti-connexin peptide or peptidomimetic.

In other embodiments, sub-therapeutically effective amounts of one or more anti-connexin polynucleotides, anti-connexin peptides or peptidomimetics, and/or gap junction modifying agents are used or provided alone or in combination to provide a desired therapeutic effect.

Compositions

Accordingly, in one aspect, the invention provides compositions for use in treating a subject during or following an orthopedic procedure or surgery or suffering from, predisposed to, or at risk for various orthopedic-related diseases, disorders, or conditions, which comprises: at least one anti-connexin polynucleotide and at least one anti-connexin peptide, peptidomimetic, or gap junction modifying agent to be administered in combination with either or both or alone. In a preferred embodiment, the composition further comprises a pharmaceutically acceptable carrier or vehicle.

In certain embodiments, compositions comprising sub-therapeutically effective amounts of one or more anti-connexin polynucleotides, anti-connexin peptides or peptidomimetics, and/or gap junction modifying agents may be used or provided alone or in combination to provide a desired therapeutic effect.

In one preferred form, the composition contains one or more antisense polynucleotides to the mRNA of one connexin protein only. In another preferred form, the composition comprises one or more anti-connexin peptides, peptidomimetics, or gap junction modifying agents (e.g. a gap junction or hemichannel phosphorylation agent or connexin carboxy-terminal polypeptide). Most preferably, this connexin protein is connexin 43.

In another preferred form, the composition comprises an anti-connexin peptide or peptidomimetic and an antisense polynucleotide to the mRNA of a connexin protein. Most preferably, this connexin is connexin 43.

The compositions may comprise anti-connexin polynucleotides or anti-connexin peptides, or other anti-connexin agents with either or both, that are directed to more than one connexin protein. Preferably, one of the connexin proteins to which polynucleotides or anti-connexin peptides or other anti-connexin agents are directed is connexin 43. Other connexins to which the polynucleotides or anti-connexin peptides or other anti-connexin agents are directed may include, for example, connexins 26, 30, 30.3, 31.1, 32, 36, 37, 40, 40.1, 44.6, 45 and 46. Suitable exemplary polynucleotides (and ODNs) directed to various connexins are set forth in Table 1. Suitable anti-connexin peptides are also provided herein. Suitable gap junction or hemichannel phosphorylation agents and connexin carboxy-terminal polypeptides are known in the art.

Kits, Medicaments and Articles of Manufacture

Optionally, an anti-connexin peptide, or one or more anti-connexin polynucleotides and one or more anti-connexin peptides, peptidomimetics, or gap junction modifying agents, such as a gap junction or hemichannel phosphorylation agent or connexin carboxy-terminal polypeptide, may also be used in the manufacture of the medicament for treating a subject during or following an orthopedic procedure or surgery or suffering from, predisposed to, or at risk for various orthopedic-related diseases, disorders, or conditions.

In one aspect, the invention provides a kit for treating a subject during or following an orthopedic procedure or surgery or suffering from, predisposed to, or at risk for various orthopedic-related diseases, disorders, or conditions comprising one or more compositions or formulations described. For example, the invention includes a kit comprising a composition comprising a therapeutically effective amount of an anti-connexin peptide or peptidomimetic, alone or in combination with one or more gap junction modifying agent. For example, the kit may include a composition comprising an effective amount of an anti-connexin peptide, or one or more anti-connexin polynucleotides and one or more anti-connexin peptides, peptidomimetics, or gap junction modifying agents, such as a gap junction or hemichannel phosphorylation agent or connexin carboxy-terminal polypeptide.

Articles of manufacture are also provided for treating a subject during or following an orthopedic procedure or surgery or suffering from, predisposed to, or at risk for various orthopedic-related diseases, disorders, or conditions, comprising a vessel containing a composition or formulation of the invention as described herein and instructions for use for the treatment of a subject. For example, in another aspect, the invention includes an article of manufacture comprising a vessel containing a therapeutically effective amount of an anti-connexin peptide or peptidomimetic, alone or in combination with one or more gap junction modifying agents. In another aspect, the invention includes an article of manufacture comprising a vessel containing a therapeutically effective amount of an anti-connexin peptide, or one or more anti-connexin polynucleotides and one or more anti-connexin peptides, peptidomimetics, or gap junction modifying agents and/or other anti-connexin agents, such as a gap junction or hemichannel phosphorylation agent or connexin carboxy-terminal polypeptide, and instructions for use, including use for the treatment of a subject during or following an orthopedic procedure or surgery or suffering from, predisposed to, or at risk for various orthopedic-related diseases, disorders, or conditions. In other embodiments, sub-therapeutically effective amounts of one or more anti-connexin polynucleotides, anti-connexin peptides or peptidomimetics, and/or gap junction modifying agents are used or provided alone or in combination in an article of manufacture to provide a desired therapeutic effect.

Treatment

The compositions and formulations of the invention may be used in conjunction or combination with a composition for treating a subject during or following an orthopedic procedure or surgery or suffering from, predisposed to, or at risk for various orthopedic-related diseases, disorders, or conditions.

In one aspect the invention is directed to a method of for treating a subject suffering from, predisposed to, or at risk for various orthopedic-related diseases, disorders, or conditions, comprising administration a therapeutically effective amount of one or more one or more anti-connexin peptides or peptidomimetics alone or in combination with one or more or gap junction modifying agents or, optionally, one or more anti-connexin polynucleotides and one or more anti-connexin peptides, peptidomimetics, or gap junction modifying agents, such as a gap junction or hemichannel phosphorylation agent or connexin carboxy-terminal polypeptide. In another aspect, sub-therapeutically effective amounts of one or more anti-connexin polynucleotides, anti-connexin peptides or peptidomimetics, and/or gap junction modifying agents are used or provided alone or in combination to provide a desired therapeutic effect.

In one embodiment the orthopedic diseases, disorders and conditions, include, for example, orthopedic diseases, disorders, and conditions including, for example, anterior cruciate ligament (acl) injury; arthritis of the shoulder; articular cartilage injury of the knee; bowlegs; broken back; broken hip (pelvis); broken leg; broken neck; bunions; bursitis; carpal tunnel syndrome; chronic low back pain; clubfoot; curvature of the spine (scoliosis); diabetic foot; dislocated elbow; dislocated hip; dupuytren's contracture; flatfoot; foot deformity; forearm fractures in children; hemophiliac arthritis; herniated disc (slipped disc); hip labral tear; hip arthritis; infectious arthritis; inflammatory hip conditions; intoeing; knee arthritis; knock knee; meniscal tear; osteoarthritis; osteonecrosis; osteoporosis; rheumatoid arthritis; rotator cuff injuries; scoliosis; shoulder arthritis; shoulder instability; shoulder pain; slipped disc (herniated disc); spinal stenosis/degenerative spondylolisthesis; spondylolisthesis; sports injuries to foot; sprained ankle; tendonitis; thumb arthritis; and trigger finger.

In one aspect the invention is directed to a method of for treating a subject following a surgical procedure, comprising administration a therapeutically effective amount of one or more one or more anti-connexin peptides or peptidomimetics alone or in combination with one or more or gap junction modifying agents or, optionally, one or more anti-connexin polynucleotides and one or more anti-connexin peptides, peptidomimetics, or gap junction modifying agents, such as a gap junction or hemichannel phosphorylation agent or connexin carboxy-terminal polypeptide. In certain embodiments, the administration is effective to post-operative joint contracture. In certain embodiments, the administration is effective to prevent or reduce contracture, in whole or in part. In certain embodiments, the administration is effective to improve recovery time in a post-operative subject. In one embodiment, administration is effective to decrease pain in a post-operative subject. In one embodiment, administration is effective to improve overall recovery result in a post-operative subject. In one embodiment, improved recovery results comprises increased post-operative mobility. In one embodiment, administration is effective to prevent or decrease vascular damage in or around the surgical site. In other embodiments, sub-therapeutically effective amounts of one or more anti-connexin polynucleotides, anti-connexin peptides or peptidomimetics, and/or gap junction modifying agents are used or provided alone or in combination to provide desired therapeutic or other effects.

In one embodiment the method comprises treatment of a subject following one of the following orthopedic procedures: an arthroscopic procedure, a joint surgery (e.g., hip, shoulder or knee surgery, including replacement procedures). In general, orthopedic surgeries addressed with the inventions described and claimed herein include hand surgery; shoulder and elbow surgery; total joint reconstruction (arthroplasty); foot and ankle surgery; spine surgery; surgical sports medicine; and orthopedic trauma. Thus, for example, orthopedic surgeries include knee arthroscopy and meniscectomy; shoulder arthroscopy and decompression; carpal tunnel release; knee arthroscopy and chondroplasty; removal of support implants; knee arthroscopy and anterior cruciate ligament reconstruction; knee replacement; repair of femoral neck fractures; repair of trochanteric fractures; debridement of skin/muscle/bone/fracture; knee arthroscopy repair of both menisci; hip replacement; shoulder arthroscopy/distal clavicle excision; repair of rotator cuff tendon; repair fracture of radius/ulna; laminectomy; repair of ankle fracture (bimalleolar type); shoulder arthroscopy and débridement; lumbar spinal fusion; repair fracture of the distal radius; low back intervertebral disc surgery; incise finger tendon sheath; repair of ankle fracture (fibula); repair of femoral shaft fracture; repair of trochanteric fracture. Total hip replacement, total shoulder replacement, and total knee replacement are included as well, as is uni-compartment knee replacement, in which only one side of an arthritic knee is replaced, and joint replacements for other joints, including elbow, wrist, ankle, and fingers. Also included in orthopedic surgeries is bone grafting, a surgical procedure that replaces missing bone with material from the patient's own body, or an artificial, synthetic, or natural substitute.

In the case of established disease, disorders or conditions, the compositions of the invention can be used to complement a release procedure (e.g., forced manipulation, open release, arthroscopic release, or debulking of scar) to prevent the recurrence abnormal tissue formation and/or further contracture formation in and/or around a joint. For example, in one embodiment of the invention, methods are provided for treating a Carpal Tunnel Syndrome (e.g., Carpal Tunnel release surgery), contracture in and/or around a joint (“joint contracture”), or recurrence thereof, comprising administering a effective amount of a composition comprising an anti-connexin agent. In on embodiment, the composition is administered to the site of the injury before, at the time of or after a release procedure.

In one aspect the invention is directed to a method of for treating a subject following a surgical procedure or suffering from, predisposed to, or at risk for various orthopedic-related diseases, disorders, or conditions, comprising administration a therapeutically effective amount of an anti-connexin peptide or peptidomimetic in combination with one or more gap junction modifying agents. In one embodiment, the anti-connexin peptide, peptidomimetic, or gap junction modifying agent is effective to improve surgical outcome. In one embodiment, the anti-connexin peptide, peptidomimetic, or gap junction modifying agent is effective to prevent or reduce contracture. In other embodiments, sub-therapeutically effective amounts of one or more anti-connexin peptides or peptidomimetics one or more gap junction modifying agents are used or provided alone or in combination to provide a desired therapeutic effect.

In one embodiment, the anti-connexin agent is a connexin antisense polynucleotide effective to downregulate or inhibit connexin protein expression. In one embodiment, the connexin antisense polynucleotide is a connexin 26 antisense polynucleotide, peptide or peptidomimetic, a connexin 43 antisense polynucleotide, peptide, or peptidomimetic or a mixture thereof.

In one aspect the invention is directed to sustained administration of an anti-connexin peptide (e.g., a hemichannel blocker such a peptidomimetic), or one or more anti-connexin polynucleotides and one or more anti-connexin peptides or peptidomimetics, or, optionally, to sustained administration of one or more anti-connexin polynucleotides and/or one or more anti-connexin peptides, peptidomimetics, or gap junction modifying agents, such as a gap junction or hemichannel phosphorylation agent or connexin carboxy-terminal polypeptide. In one embodiment, the anti-connexin agents are administered for at least about 0.5 hours, about 1-24 hours, at least about 2, hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours or at least about 24 hours. In one embodiment, connexin expression is downregulated over a sustained period of time. In another embodiment, connexin hemichannels are blocked or closed, in whole or in part, over a preferred period of time. Preferably connexin 43 expression is downregulated or inhibited and connexin hemichannel opening is blocked or inhibited, in whole or in part, for a sustained period of time. Conveniently, connexin 43 expression is downregulated or inhibited or hemichannels blocked or inhibited for at least about 1, 2, 4, 6, 8, 10, 12, or 24 hours.

When not administered as a fixed combination, preferred methods include the sequential administration of one or more anti-connexin polynucleotides and one or more anti-connexin peptides or peptidomimetics, or, optionally, one or more anti-connexin polynucleotides and/or one or more anti-connexin peptides, peptidomimetics, or gap junction modifying agents, such as a gap junction or hemichannel phosphorylation agent or connexin carboxy-terminal polypeptide. Preferably, the agents are administered sequentially within at least about one-half hour of each other. The agents may also be administered with about one hour of each other, with about one day to about one week of each other, or as otherwise deemed appropriate. Preferably, an anti-connexin peptide or anti-connexin peptidomimetic, e.g., an anti-connexin agent that can block or reduce hemichannel opening, is administered prior to the administration of an anti-connexin agent that blocks or reduces connexin expression or the formation of hemichannels or gap junctions, e.g., by downregulation of connexin protein expression. Preferably, the anti-connexin agent or agents is/are anti-connexin 43 agent(s).

In another embodiment for treating a subject during or following an orthopedic procedure or surgery or suffering from, predisposed to, or at risk for various orthopedic-related diseases, disorders, or conditions, either or both of the one or more anti-connexin polynucleotides and one or more anti-connexin peptides or peptidomimetics, or, optionally, one or more anti-connexin polynucleotides and/or one or more anti-connexin peptides, peptidomimetics, or gap junction modifying agents, such as a gap junction or hemichannel phosphorylation agent or connexin carboxy-terminal polypeptide, are provided in amounts or doses that are less that those used when the agent or agents are administered alone, i.e., when they are not administered in combination. Such lesser amounts of agents administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone. Subjects which may be treated include mammals, preferably humans.

In one embodiment the method for treating a subject during or following an orthopedic procedure or surgery or suffering from, predisposed to, or at risk for various orthopedic-related diseases, disorders, or conditions comprises sustained administration of an anti-connexin peptide, peptidomimetic or gap junction modifying agent, or, optionally, one or more anti-connexin polynucleotides and one or more anti-connexin peptides or peptidomimetics or, optionally, one or more anti-connexin polynucleotides and/or one or more anti-connexin peptides, peptidomimetics, or gap junction modifying agents, such as a gap junction or hemichannel phosphorylation agent or connexin carboxy-terminal polypeptide. In one embodiment, the composition or compositions are administered in a sustained release formulation. In another embodiment, the composition or compositions are administered for a sustained period of time. Conveniently, the composition is effective to decrease connexin 43 levels, or block or reduce connexin 43 hemichannel opening, for at least about 1-2 hours, about 2-4 hours, about 4-6 hours, about 4-8 hours, about 12 hours, about 18 hours, or about 24 hours. Subjects which may be treated include mammals, preferably humans.

The following examples which will be understood to be provided by way of illustration only and not to constitute a limitation on the scope of the invention.

### EXAMPLES

### Example 1

### Maximum Tolerated Dose Determination by Intra-Articular Injection

Methods of sequentially administering anti-connexin 43 peptide preparation prepared with the following exemplary sequence: SRPTEKTIFII (SEQ.ID.NO.:19) followed by administration of an anti-connexin 43 polynucleotide preparation prepared with the following exemplary sequences: GTA ATT GCG GCA GGA GGA ATT GTT TCT CTC (connexin 43) (SEQ.ID.NO:2) and GAC AGA AAC AAT TCC TCC TGC CGC ATT TAC (sense control) (SEQ.ID.NO:7) are evaluated for the efficacy in the treatment of orthopedic related conditions.

Male Hartley guinea pigs, at least 6 weeks old, are anesthetized using 5% isoflurane in an enclosed chamber. The animals are weighed and then transferred to the surgical table where anesthesia is maintained by nose cone with 2% isoflurane. The knee area on both legs are shaved and knee width at the head of the femur is measured on both knees. The skin on the right knee is sterilized. A 25 G needle is introduced into the synovial cavity using a medial approach and 0.1 ml of the test formulations is injected. Seven days after the injection, the animals are sacrificed by cardiac injection of 0.7 ml Euthanyl under deep anesthesia (5% isoflurane).

Assessment of tolerability: Knee function is assessed before sacrifice by recording changes in walking behavior and signs of tenderness. The animal is weighed immediately after sacrifice. The width of both knees at the head of the femur is then measured with calipers. The knee joint is dissected open by transecting the quadriceps tendon, cutting through the lateral and medial articular capsule and flipping the patella over the tibia. Knee inflammation is assessed by recording signs of swelling, vascularization, fluid accumulation and change in color in subcutaneous tissue as well as inner joint structures.

### Example 2

### Spinal Surgery Model

The rabbit laminectomy spinal adhesion model described herein is used to investigate prevention of spinal scarring and abnormal tissue formation (e.g. adhesion) by local administration of an anti-connexin agent. Five to six animals will be included in each experimental group to allow for meaningful statistical analysis. Formulations with various concentrations of anti-connexin agent are tested against control animals to assess inhibition of scarring and adhesion formation.

Rabbits are anesthetized with an IM injection of ketamine/zylazine. An endotracheal tube is inserted for maintenance of anesthesia with halothane. The animal is placed prone on the operating table on top of a heating pad and the skin over the lower half of the back is shaved and prepared for sterile surgery. A longitudinal midline skin incision is made from L-1 to L-5 and down the lumbosacral fascia. The fascia is incised to expose the tips of the spinous processes. The paraspinous muscles are dissected and retracted from the spinous process and lamina of L-4. A laminectomy is performed at L-4 by removal of the spinal process with careful bilateral excision of the laminae, thus creating a small 5×10 mm laminectomy defect. Hemostasis is obtained with Gelfoam. The test formulations are applied to the injury site and the wound is closed in layers with Vicryl sutures. The animals are placed in an incubator until recovery from anesthesia and then returned to their cage.

Two weeks after surgery, the animals are anesthetized using procedures similar to those described above. The animals are euthanized with Euthanyl. After a skin incision, the laminectomy site is analyzed by dissection and the amount of scarring and adhesion is scored using scoring systems published in the scientific literature for this type of injury.

### Example 3

### Tendon Surgery Model

This model is used to the prevention of tendon damage can be prevented by localized sequential administration of anti-connexin peptides and anti-connexin polynucleotides. Extended release formulations are loaded with drugs, preferably one or more anti-connexin 43 agents, and implanted around injured tendons in rabbits.

Rabbits are anesthetized and the skin over the right hind limb is shaved and prepared for sterile surgery. Sterile surgery is performed aided by an operating microscope. A longitudinal midline skin incision is made on the volvar aspect of the proximal phalange in digits 2 and 4. The synovial sheath of the tendons is carefully exposed and incised transversally to access the flexor digitorum profundus distal to the flexor digitorum superficialis bifurcation. Tendon injury is performed by gently lifting the flexor digitorum profundus with curved forceps and incising transversally through half of its substance. The test formulations are applied around the tendons in the sheath of one of the two digits randomly selected. The other digit is left untreated and is used as a control. The sheath is then repaired with 6-0 nylon suture. An immobilizing 6-0 nylon suture is inserted through the transverse metacarpal ligament into the tendon/sheath complex to immobilize the tendon and the sheath as a single unit. The wound is closed with 4-0 interrupted sutures. A bandage is applied around the hind paw to further augment immobilization of the digits and ensure comfort and ambulation of the animals. The animals are recovered and returned to their cage.

Three weeks after surgery, the animals are anesthetized. After a skin incision, the tissue plane around the synovial sheath is dissected and the tendon-sheath complex harvested en block and transferred in 10% phosphate buffered formaldehyde for histopathology analysis. The animals are then euthanized. After paraffin embedding, serial 5 μm thin cross-sections are cut every 2 mm through the sheath and tendon complex. Sections are stained with H&E and Movat's stains to evaluate adhesion growth. Each slide is digitized using a computer connected to a digital microscope camera (Nikon Micropublisher cooled camera). Morphometry analysis is then performed using image analysis software (ImagePro). Thickness and area of adhesion defined as the substance obliterating the synovial space are measured and compared between formulation-treated and control animals.

### Example 4

Anti-connexin agent is conveniently formulated in a form suitable for administration according to the methods of the present invention.

Suitable formulations include a mixture of the following formulating agents. The amount of the individual anti-connexin agent or agents and formulating agents will depend on the particular use intended.

ASO in PBS
Polyquarternium 10
HEC/HPMC/CMC
Na Hyaluronate
Tween 20
Poloxamer 188
Plutonic 87 NF
SLES
Poly L-lysine/Polyethylene Imine
Banzalkonium chloride
Methyl paraben
Propl paraben
Propylene Glycol
10 mM Phosphate Buffer

### Example 5

Formulations for use according to methods of the present invention are prepared by mixing the compounds in the proportions noted below. In one preferred embodiment, the anti-connexin agent is an anti-connexin polynucleotide. In other embodiments, the anti-connexin polynucleotide is an anti-sense oligonucleotide, for example, an antisense oligonucleotide of SEQ.ID.NO.1.

### Formulation A

Made up of the following materials (% w/w)—Anti-connexin agent in phosphate-buffered saline (0.47%); Methylparaben (0.17%); Propylparaben (0.03%); Propylene Glycol (1.5%); HPMC (1.5%); and 10 mM Phosphate Buffer (96.33%). Formulation is a clear gel with pH ˜6.74 and osmolality of 244.

### Formulation B

Made up of the following materials (% w/w)—Anti-connexin agent in phosphate-buffered saline (0.47%); Methylparaben (0.17%); Propylparaben (0.03%); Propylene Glycol (1.5%); HPMC (1.5%); 0.5% BAC (0.1%); and 10 mM Phosphate Buffer (96.23%). Formulation is a clear gel with pH ˜6.65 and osmolality of 230.

### Formulation C

Made up of the following materials (% w/w)—Anti-connexin agent in phosphate-buffered saline (0.47%); Methylparaben (0.17%); Propylparaben (0.03%); Propylene Glycol (1.5%); HPMC (1.5%); Polyquaternium 10 (0.5%); Poloxamer 188 (0.1%); and 10 mM Phosphate Buffer (95.73%). Formulation is a slightly hazy gel with pH ˜6.59 and osmolality of 233.

### Formulation D

Made up of the following materials (% w/w)—Anti-connexin agent in phosphate-buffered saline (0.47%); Methylparaben (0.17%); Propylparaben (0.03%); Propylene Glycol (1.5%); HPMC (1.5%); SLES (0.5%); and 10 mM Phosphate Buffer (95.83%). Formulation is a clear gel with pH ˜6.8 and osmolality of 246.

### Formulation E

Made up of the following materials (% w/w)—Anti-connexin agent in phosphate-buffered saline (0.47%); Methylparaben (0.17%); Propylparaben (0.03%); Propylene Glycol (1.5%); HPMC (1.5%); Poloxamer 188 (0.1%); 25K Polyethylene Imine (0.075%); and 10 mM Phosphate Buffer (96.155%). Formulation is a hazy gel with pH ˜7.8 and osmolality of 249.

### Formulation F

Made up of the following materials (% w/w)—Anti-connexin agent in phosphate-buffered saline (0.47%); Methylparaben (0.17%); Propylparaben (0.03%); Propylene Glycol (1.5%); HPMC (1.5%); Sodium Hyaluronate (0.1%); and 10 mM Phosphate Buffer (96.23%). Formulation is a clear gel with pH ˜6.88 and osmolality of 289.

### Formulation G

Made up of the following materials (% w/w)—Anti-connexin agent in phosphate-buffered saline (0.47%); Methylparaben (0.17%); Propylparaben (0.03%); Propylene Glycol (1.5%); Sodium Hyaluronate (1.0%); and 10 mM Phosphate Buffer (96.83%). Formulation is a clear gel with pH ˜6.81 and osmolality of 248.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1. A method of treating a subject undergoing an orthopedic procedure comprising administration of a composition comprising a therapeutically effective amount of a first anti-connexin agent and a second anti-connexin agent to a site of injury within said subject before, at the time of, or after said orthopedic procedure, wherein said first agent is an anti-connexin polynucleotide agent and said second agent is an anti-connexin peptide or peptidomimetic.

2. A method of claim 1, wherein a surgical outcome is improved.

3. A method of claim 2, wherein the surgical outcome is improved recovery time, reduced pain and/or improved mobility.

4. A method according to claim 1, wherein said polynucleotide is an antisense polynucleotide.

5. A method according to claim 4, wherein said antisense polynucleotide comprises a sequence selected from SEQ.ID.NOS:1 to 12.

6. A method according to claim 4, wherein said antisense polynucleotide is selected from: GTA ATT GCG GCA AGA AGA ATT GTT TCT GTC (SEQ ID NO:1); GTA ATT GCG GCA GGA GGA ATT GTT TCT GTC (SEQ ID NO:2); and, GGC AAG AGA CAC CAA AGA CAC TAC CAG CAT (SEQ ID NO:3).

7. A method according to claim 4, wherein said antisense polynucleotide has from about 15 to about 35 nucleotides and is sufficiently complementary to connexin 43 mRNA to form a duplex having a melting point greater than 20° C. under physiological conditions.

8. A method according to claim 4, wherein the antisense polynucleotide has from about 15 to about 35 nucleotides and has at least about 70 percent homology to an antisense sequence of connexin 43 mRNA.

9. A method according to claim 1, wherein the composition comprises about 0.1 to about 1.000 micrograms of said anti-connexin agent and the anti-connexin 43 agent is an antisense polynucleotide.

10. A method of claim 1, wherein said peptide comprises a sequence selected from SEQ.ID.NOS:15 to 23.

11. A method according to claim 1, wherein the composition comprises about 0.01 to about 100 milligrams of said anti-connexin 43 peptide or anti-connexin 43 peptidomimetic.

12. A method according to claim 1, wherein said anti-connexin polynucleotide agent is an RNAi or siRNA polynucleotide.

13. A method according to claim 1, wherein the subject is a mammal.

14. A method according to claim 13, wherein the mammal is a human.

15. A method according to claim 13, wherein the mammal is selected from the group consisting of domestic animals, farm animals, zoo animals, sports animals, and pets.

16. A method according to claim 15, wherein the mammal is a horse.

17. A method according to claim 15, wherein the mammal is a dog or a cat.

18. A method of decreasing pain in a post-orthopedic surgical subject comprising administration of a first composition and a second composition, said first composition comprising a therapeutically effective amount of an anti-connexin 43 polynucleotide and said second composition comprising a therapeutically effective amount of an anti-connexin 43 peptide or peptidomimetic.

19. A method of claim 18, wherein a surgical outcome is improved.

20. A method of claim 19, wherein the surgical outcome is improved recovery time, reduced pain and/or improved mobility.

21. A method according to claim 18, wherein the first and second compositions are administered simultaneously.

22. A method according to claim 18, wherein the first and second compositions are administered within at least about one hour of each other.

23. A method according to claim 18, wherein first and second compositions are administered within about 5 minutes of each other, within about 10 minutes of each other, within about 15 minutes of each other, or within about 30-90 minutes of each other.

24. A method according to claim 18, wherein the first composition is administered first.

25. A method according to claim 18, wherein the second composition is administered first.

26. A method according to claim 18, further comprising administration of a third composition, wherein the third composition comprises an anti-connexin polynucleotide, anti-connexin peptide, anti-connexin peptidomimetic, or gap junction modifying agent.

27. A method according to claim 18, wherein the third composition is administered first.

28. A method according to claim 18, wherein said polynucleotide is an antisense polynucleotide.

29. A method according to claim 28, wherein said antisense polynucleotide comprises a sequence selected from SEQ.ID.NOS:1 to 12.

30. A method according to claim 28, wherein said antisense polynucleotide is selected from: GTA ATT GCG GCA AGA AGA ATT GTT TCT GTC (SEQ ID NO:1); GTA ATT GCG GCA GGA GGA ATT GTT TCT GTC (SEQ ID NO:2); and, GGC AAG AGA CAC CAA AGA CAC TAC CAG CAT (SEQ ID NO:3).

31. A method according to claim 28, wherein said antisense polynucleotide has from about 15 to about 35 nucleotides and is sufficiently complementary to connexin 43 mRNA to form a duplex having a melting point greater than 20° C. under physiological conditions.

32. A method according to claim 28, wherein the antisense polynucleotide has from about 15 to about 35 nucleotides and has at least about 70 percent homology to an antisense sequence of connexin 43 mRNA.

33. A method according to claim 18, wherein the composition comprises about 0.1 to about 1000 micrograms of said anti-connexin agent and the anti-connexin 43 agent is an antisense polynucleotide.

34. A method of claim 18, wherein said peptide comprises a sequence selected from SEQ.ID.NOS:15 to 23.

35. A method according to claim 18, wherein the composition comprises about 0.01 to about 100 milligrams of said anti-connexin 43 peptide or anti-connexin 43 peptidomimetic.

36. A method according to claim 18, wherein said anti-connexin polynucleotide is an RNAi or siRNA polynucleotide.

37. A method according to claim 18, wherein the subject is a mammal.

38. A method according to claim 37, wherein the mammal is a human.

39. A method according to claim 37, wherein the mammal is selected from the group consisting of domestic animals, farm animals, zoo animals, sports animals, and pets.

40. A method according to claim 39, wherein the mammal is a horse.

41. A method according to claim 39, wherein the mammal is a dog or a cat.

42. A method of decreasing pain in a post-orthopedic-surgical subject comprising administration of one or more anti-connexin peptides or peptidomimetics and one or more anti-connexin polynucleotides during or at the conclusion of said surgery.

43. A method according to claim 42, wherein said polynucleotide is an antisense polynucleotide.

44. A method according to claim 43, wherein said antisense polynucleotide comprises a sequence selected from SEQ.ID.NOS:1 to 12.

45. A method according to claim 43, wherein said antisense polynucleotide is selected from: GTA ATT GCG GCA AGA AGA ATT GTT TCT GTC (SEQ ID NO:1); GTA ATT GCG GCA GGA GGA ATT GTT TCT GTC (SEQ ID NO:2); and, GGC AAG AGA CAC CAA AGA CAC TAC CAG CAT (SEQ ID NO:3).

46. A method according to claim 43, wherein said antisense polynucleotide has from about 15 to about 35 nucleotides and is sufficiently complementary to connexin 43 mRNA to form a duplex having a melting point greater than 20° C. under physiological conditions.

47. A method according to claim 43, wherein the antisense polynucleotide has from about 15 to about 35 nucleotides and has at least about 70 percent homology to an antisense sequence of connexin 43 mRNA.

48. A method according to claim 42, wherein the composition comprises about 0.1 to about 1000 micrograms of said anti-connexin polynucleotide and the anti-connexin 43 polynucleotide is an antisense polynucleotide.

49. A method of claim 42, wherein said peptide comprises a sequence selected from SEQ.ID.NOS:15 to 23.

50. A method according to claim 42, wherein the composition comprises about 0.01 to about 100 milligrams of said anti-connexin 43 peptide or anti-connexin 43 peptidomimetic.

51. A method according to claim 42, wherein said anti-connexin polynucleotide is an RNAi or siRNA polynucleotide.

52. A method according to claim 42, wherein the subject is a mammal.

53. A method according to claim 52, wherein the mammal is a human.

54. A method according to claim 52, wherein the mammal is selected from the group consisting of domestic animals, farm animals, zoo animals, sports animals, and pets.

55. A method according to claim 54, wherein the mammal is a horse.

56. A method according to claim 54, wherein the mammal is a dog or a cat.

57. A pharmaceutical composition for use in treating a subject during or following an orthopedic procedure or surgery, which comprises therapeutically effective amounts of an anti-connexin 43 polynucleotide and an anti-connexin 43 peptide or peptidomimetic.

58. A pharmaceutical composition according to claim 57, wherein said polynucleotide is an antisense polynucleotide.

59. A pharmaceutical composition according to claim 58, wherein said antisense polynucleotide comprises a sequence selected from SEQ.ID.NOS:1 to 12.

60. A pharmaceutical composition according to claim 58, wherein said antisense polynucleotide is selected from: GTA ATT GCG GCA AGA AGA ATT GTT TCT GTC (SEQ ID NO:1); GTA ATT GCG GCA GGA GGA ATT GTT TCT GTC (SEQ ID NO:2); and, GGC AAG AGA CAC CAA AGA CAC TAC CAG CAT (SEQ ID NO:3).

61. A pharmaceutical composition according to claim 58, wherein said antisense polynucleotide has from about 15 to about 35 nucleotides and is sufficiently complementary to connexin 43 mRNA to form a duplex having a melting point greater than 20° C. under physiological conditions.

62. A pharmaceutical composition according to claim 58, wherein the antisense polynucleotide has from about 15 to about 35 nucleotides and has at least about 70 percent homology to an antisense sequence of connexin 43 mRNA.

63. A pharmaceutical composition according to claim 58, wherein the composition comprises about 0.1 to about 1000 micrograms of said anti-connexin agent and the anti-connexin 43 agent is an antisense polynucleotide.

64. A pharmaceutical composition of claim 57, wherein said peptide comprises a sequence selected from SEQ.ID.NOS:15 to 23.

65. A pharmaceutical composition according to claim 57, wherein the composition comprises about 0.01 to about 100 milligrams of said anti-connexin 43 peptide or anti-connexin 43 peptidomimetic.

66. A pharmaceutical composition according to claim 57, wherein said anti-connexin agent is an RNAi or siRNA polynucleotide.

67. A pharmaceutical composition according to claim 57 which is formulated for topical administration.

68. A pharmaceutical composition according to claim 57 which is formulated as a gel.

69. A pharmaceutical composition according to claim 57, wherein said gel is a polyoxyethylene-polyoxypropylene copolymer-based gel or a carboxymethylcellulose-based gel.

70. A pharmaceutical composition according to claim 57, wherein said gel is a pluronic gel.

71. A pharmaceutical composition according to claim 58, effective to prevent or decrease pain in a post-operative subject.

72. A pharmaceutical composition according to claim 58, effective to prevent or decrease vascular damages associated with an orthopedic surgery or procedure.

73. A pharmaceutical composition according to claim 58, effective to improve surgical outcome.

74. A pharmaceutical composition according to claim 58, effective to improve overall recovery from an orthopedic surgery or procedure.

75. An article of manufacture comprising package material containing a pharmaceutical composition according to claim 57 together with instructions for use in or on a subject during or following an orthopedic procedure or surgery.

76. A method of treating a subject undergoing an orthopedic procedure, comprising administration of a composition comprising administration a therapeutically effective amount of an anti-connexin peptide, alone or in combination with one or more of an anti-connexin oligonucleotide, a hemichannel phosphorylation compound, and a connexin carboxy-terminal peptide for inhibition of ZO-1 protein interaction to a site of injury within said subject before, at the time of or after said orthopedic procedure, wherein a surgical outcome is improved.

77. A method according to claim 76, wherein the connexin is connexin 43.

78. A method of treating a subject undergoing an orthopedic procedure, comprising administration of a composition comprising amount of an anti-connexin peptide sufficient to improve post-operative pain, mobility and/or recovery time, alone or in combination with one or more of an anti-connexin oligonucleotide for downregulation of connexin protein expression, a hemichannel phosphorylation compound for hemichannel closing, and a connexin carboxy-terminal peptide for inhibition of ZO-1 protein interaction to a site of injury within said subject before, at the time of, or after said orthopedic procedure, wherein a surgical outcome is improved.

79. A method according to claim 78, wherein the connexin is connexin 43.

80. A method of treating and/or preventing, in whole or in part, post-orthopedic surgical joint contracture in a subject comprising administering a composition comprising a first composition comprising an effective amount of an anti-connexin 43 polynucleotide and a second composition comprising a therapeutically effective amount of a connexin 43 peptide or peptidomimetic to said subject during or after said surgery.
